(19) United States
(12) Patent Application Publication
(10) Pub. No.: US 2017/0191031 A1
(43) Pub. Date: Jul. 6, 2017

(54) SYSTEMS AND METHODS FOR STEM CELL DIFFERENTIATION

(71) Applicant: University of Oslo, Oslo (NO)

(72) Inventors: Gareth Sullivan, Oslo (NO); Sebastian Greenhough, Oslo (NO)

(21) Appl. No.: 15/304,884

(22) PCT Filed: Apr. 30, 2015
(86) PCT No.: PCT/IB2015/001229

§ 371 Date: Oct. 18, 2016

Related U.S. Application Data
(60) Provisional application No. 61/986,489, filed on Apr. 30, 2014.

(51) Int. Cl. C12N 5/071

(52) U.S. Cl. CPC C12N 5/067; C12N 5/0672; C12N 2501/727; C12N 2506/45; C12N 2506/02; C12N 2501/12; C12N 2501/39; C12N 2501/999; C12N 2500/62

(57) ABSTRACT

The present invention relates to compositions and method for differentiating stem cells. In particular, the present invention relates to methods of generating hepatocytes from human pluripotent stem cells (hPSCs) using a small molecule-driven approach.

### CROSS-REFERENCE TO RELATED APPLICATION

The present application claims priority to pending U.S. Provisional Patent Application No. 61/986,489, filed Apr. 30, 2014, the contents of which are incorporated by reference in its entirety.

### FIELD OF THE INVENTION

The present invention relates to compositions and method for differentiating stem cells. In particular, the present invention relates to methods of generating hepatocytes from human pluripotent stem cells (hPSCs) using a small molecule-driven approach.

### BACKGROUND OF THE INVENTION

Human embryonic stem cells (hESCs) and induced pluripotent stem cells (hiPSCs) offer a potentially limitless source of cells for industrial and clinical translation, and the ever-advancing field of cellular reprogramming has redefined the limits of cell plasticity (Taylor J, et al., Cell Res 2010; 20:502-3). Meanwhile cell culture and production technologies are rapidly improving and the first instances of pluripotent cell-derived therapies have entered clinical trials (Schwartz S D, et al., Lancet 2012; 379:713-20).

Amidst this broadening field, hepatocyte generation is gathering interest with industrial and clinical parties due to its relevance in the areas of drug development, cell therapy and disease modelling. While the scope of what could be achieved is wide, a number of obstacles remain before human pluripotent stem cell (hPSC) technologies can be adopted for widespread use. On the one hand are the technical challenges relating to scalability, definition and reproducibility, while from a basic research perspective many unanswered questions remain regarding the development of an adult phenotype, the mechanisms of cell reprogramming and the role of the tissue culture microenvironment.

Early protocols for the generation of hepatocytes from pluripotent cells relied on the use of embryoid body formation (Imamura T, et al., Tissue Eng 2004; 10:1716-724; Basma H, et al. Gastroenterology 2009; 136:990-99; Baharvand H, et al., Int J Dev Biol 2006; 50:645-52). This method involves the creation of cell aggregates and the spontaneous differentiation of the pluripotent population to a mixed population of cells representing the three germ layers (Itskovitz-Eldor J. et al., Mol Med 2000; 6:88-95). Notable improvements in efficiency and functionality have since been achieved by various groups which based their protocols on developmental signalling and utilized adherent culture conditions. However, all directed differentiation protocols for hepatocyte like cells (HLCs) published to date have relied on the use of recombinant growth factors such as activin A, Wnt3a, hepatocyte growth factor (HGF), oncostatin M (OSM), fibroblast growth factor 4 (FGF-4), vascular endothelial growth factor (VEGF), epidermal growth factor (EGF) and bone morphogenetic protein 4 (BMP-4) (Hay D C, et al., PNAS USA, 2008; 105:12301-6; Agarwal S, et al. Stem Cells 2008; 26:1117-27; Brolen G, et al., J Biotechnol 2010; 145:284-94; Touboul T, et al., Hepatology 2010; 51:1754-65; Si-Tayeb K, et al., Hepatology 2010; 51:297-305; Sullivan G J, et al., Hepatology 2010; 51:329-35; Cai J, et al., Hepatology 2007; 45:1229-39; Liu H, et al., Hepatology 2010; 51:1810-9; Song Z, et al., Cell Res 2009; 19:1233-42; Chen Y F, et al., Hepatology 2012; 55:1193-203).

Some recent progress has been made in replacing growth factors for the differentiation of mesoderm and ectoderm (Lian X, et al., PNAS USA, 2012; 109:E1848-5; Chambers S M, et al., Nat Biotechnol 2012; 30:715-720), and efforts have been undertaken to find suitable candidates for the production of definitive endoderm (DE), exemplified by the Melton group who identified IDE1 and 2 (Borowiak M, et al., Cell Stem Cell 2009; 4:348-358). To date however, further endodermal differentiation has only been performed in combination with other recombinant growth factors (see Review (Han S, et al., J Stem Cell Res Ther 2012; doi:10.4172/2157-7633.S10-008)).

Additional methods that do not utilize growth factors for differentiation are needed.

### SUMMARY OF THE INVENTION

The present invention relates to compositions and method for differentiating stem cells. In particular, the present invention relates to methods of generating hepatocytes from human pluripotent stem cells (hPSCs) using a small molecule-driven approach.

For example, in some embodiments, the present disclosure provides a method of differentiating pluripotent stem cells, comprising: sequentially contacting pluripotent stem cells (e.g., in the following order) with a GSK-3 inhibitor (e.g., CHIR99021, BIO or Wnt3a); DMSO or a DMSO mimetic; and a glucocorticoid (e.g., dexamethasone or hydrocortisone 21-hemisuccinate) and/or an HGF mimetic (e.g., Nle1-AngIV; N-Acetyl-Nle-Tyr-Ile-His; D-Nle-Tyr-Ile; GABA-Tyr-Ile; Nle-Tyr-Ile-His-NH2; D-Nle-X-Ile-NH—(CH2)5-CONH2, where X is any amino acid; Nle1-Tyr2-Ile3-His4-Pro5, Nle1-Tyr2-Ile3-His4, or and Nle1-Tyr2-Ile3). In some embodiments, DEX and Dihexa are administered at a concentration of 1-1000 nm (e.g., 10-100 nm). In some embodiments, the CHIR99021 is contacted with the pluripotent stem cells for approximately 6-120 hours. In some embodiments, the DMSO is contacted with the pluripotent stem cells for approximately 2-7 days. In some embodiments, the pluripotent stem cells are for example, human embryonic stem cells or induced pluripotent stem cells (other pluripotent cells are specifically contemplated). In some embodiments, the method differentiates the pluripotent stem cells into hepatocytes.

In some embodiments, the present invention provides a kit, comprising: a GSK-3 inhibitor (e.g., CHIR99021, BIO or Wnt3a); DMSO or a DMSO mimetic; and a glucocorticoid (e.g., dexamethasone or hydrocortisone 21-hemisuccinate) and/or an HGF mimetic (e.g., Nle1-AngIV; N-Acetyl-Nle-Tyr-Ile-His; D-Nle-Tyr-Ile; GABA-Tyr-Ile; Nle-Tyr-Ile-His-NH2; D-Nle-X-Ile-NH—(CH2)5-CONH2, where X is any amino acid; Nle1-Tyr2-Ile3-His4-Pro5, Nle1-Tyr2-Ile3-His4, or and Nle1-Tyr2-Ile3). In some embodiments, components a); b); and c) are provided in separate containers.

Further embodiments provide a cell (e.g., hepatocyte, pancreas (e.g. insulin producing cells), lung, colon, and intestinal cell types (foregut & hindgut), and liver) differentiated by above described method.

Additional embodiments provide the use of the cells (e.g., hepatocytes) differentiated by the above methods in a variety of research, screening, clinical, and therapeutic applications. For example, in some embodiments, hepatocytes are utilized for drug screening and development (e.g., drug toxicity screening, assessing drug metabolism, and gentoxicity (e.g., using genetically defined lines for personalized therapy)). In some embodiments, hepatocytes find use in clinical and therapeutic applications (e.g., creating bioartifical liver devices, cell therapy (e.g., engraftment of artificially derived cells), and clinical grade cell production for therapeutics. In some embodiments, hepatocytes find use in disease modeling (e.g., use of derived cells for engraftment in animal models).

Additional embodiments are described herein.

### DESCRIPTION OF THE FIGURES

FIG. 1 shows a schematic of the differentiation process A) The normal process of differentiation and the phases of the protocol to which these apply. B) Summary of the base media, timecourse and small molecule additions for each phase of differentiation. C) Key markers expressed at each stage of differentiation. D) Morphology of H1 cells observed at key stages of differentiation using phase contrast microscopy (10×). Scale bars=100 μm.

FIG. 2 shows characterization of Phase I (definitive endoderm) differentiation A) Gene expression changes during Phase I of differentiation as measured by TaqMan RT-qPCR. B) Morphology at days 0, 1 and 2, taken using phase contrast microscopy (10×). Scale bars=100 μm. C) Expression of FOXA2 and SOX17 at Phase I endpoint, imaged using fluorescent microscopy. Scale bars=100 μm.

FIG. 3 shows characterization of Phase II (hepatic specification) differentiation A) Expression of AFP and HNF4α at Phase II endpoint, imaged using fluorescent microscopy. Scale bars=100 μm. B) Morphology at Phase II endpoint, photographed using phase contrast microscopy at 10× and 20×. Scale bars=100 μm. C) Expression of AFP and HNF4A at Phase II endpoint following both growth factor and small molecule treatments, as measured by TaqMan RT-qPCR. Normalised to β-Actin and undifferentiated control.

FIG. 4 shows characterization of phase III (hepatocyte like cells) differentiation: Morphology, RT-PCR and immunofluorescence A) Morphology at protocol endpoint, taken using phase contrast microscopy at 10× and 20×. Scale bars=100 μm. B) Expression of albumin and HNF4α at protocol endpoint, imaged using fluorescent microscopy. Scale bars=100 μm. C) Expression of AFP at protocol endpoint, imaged using fluorescent microscopy. Scale bars=100 μm. D) Expression of A1AT at protocol endpoint, imaged using fluorescent microscopy. Scale bars=100 μm. E) RT-PCR panel showing expression of hepatic markers in H1 derived HLCs.

FIG. 5 shows characterization of Phase III (hepatocyte like cells) differentiation: RT-qPCR and functional analysis A) Expression of ALB, A1AT (SERPINA1), CYP3A4 and HNF4A at endpoint of growth factor and small molecule protocols, as measured by TaqMan RT-qPCR. B) Glycogen storage in growth factor and small molecule differentiated cells as indicated by periodic acid—Schiff staining. Scale bars=100 μm. C) Cytochrome P450 (CYP) activity (1A2, 2D6, 3A4) in differentiated cells at endpoint of both growth factor and small molecule based protocols. D) Serum protein secretion at endpoint of both growth factor and small molecule based protocols.

FIG. 6 shows phase I characterization: Comparison to other small molecules and growth factors A) Expression of FOXA2 and SOX17 at Phase I endpoint following treatment with small molecule (SM), growth factor (GF) or vehicle (Veh) protocols. Imaged using fluorescent microscopy. Scale bars=100 μm. B) Expression of FOXA2 and SOX17 at Phase I endpoint following treatment with alternative GSK-3 inhibitor (BIO—1 μM). Imaged using fluorescent microscopy. Scale bars=100 μm. C) Expression of FOXA2 and SOX17 at Phase I endpoint following treatment with Wnt3a alone (50 ng/ml). Imaged using fluorescent microscopy. Scale bars=100 μm.

FIG. 7 shows a phase I 48 hour time course to assess development trajectory by RT-qPCR.

FIG. 8 shows characterisation of growth factor based hepatocyte differentiation at Phases II and III: Morphology and immunofluorescence A) Expression of AFP and HNF4α at phase II endpoint, imaged using fluorescent microscopy. Scale bars=100 μm. B) Expression of AFP at protocol endpoint, imaged using fluorescent microscopy. Scale bars=100 μm. C) Expression of albumin and HNF4α at protocol endpoint, imaged using fluorescent microscopy. Scale bars=100 μm. D) Morphology at protocol endpoint, taken using phase contrast microscopy at 20×. Scale bars=100 μm. E) Expression of A1AT at protocol endpoint, imaged using fluorescent microscopy. Scale bars=100 μm.

FIG. 9 shows verification of CYP7A1 expression by RT-PCR A) RT-PCR panel of gene expression of H1 derived HLCs. Lanes 1-3 top panel=H1 day 17 HLCs CYP7A1 expression, lower panel=β-Actin loading control; B) RT-PCR panel of gene expression of Detroit A derived HLCs. Lane 1-3 top panel=Detroit A day 17 HLCs CYP7A1 expression, lower panel=β-Actin loading control.

FIG. 10 shows characterization of smHLC differentiation of hESC line 207 A) RT-PCR panel demonstrating hepatic gene expression in 207 derived smHLCs. B) Expression of albumin, HNF4α and A1AT at protocol endpoint, imaged using fluorescent microscopy. Scale bars=100 μm. C) Upper panel, morphology at protocol endpoint, taken using phase contrast microscopy at 20×. Scale bars=100 μm. Lower panel, glycogen storage in growth factor and small molecule differentiated cells as indicated by periodic acid—Schiff staining. Scale bars=100 μm. D) Expression of ALB, A1AT (SERPINA1) and HNF4A at endpoint of protocol, as measured by TaqMan RT-qPCR. Normalised to β-actin and undifferentiated control. E) Serum protein secretion at endpoint of small molecule based protocol.

FIG. 11 shows characterization of smHLC differentiation of hiPSC line Detroit A. A) RT-PCR panel demonstrating hepatic gene expression in Detroit A derived smHLCs. B) Expression of albumin, HNF4α and A1AT at protocol endpoint, imaged using fluorescent microscopy. Scale bars=100 μm. C) Expression of ALB, A1AT (SERPINA1) and HNF4A at endpoint of protocol, as measured by TaqMan RT-qPCR. Normalised to β-actin and undifferentiated control. D) Serum protein secretion at endpoint of small molecule based protocol.

FIG. 12 shows characterization of smHLC differentiation of hiPSC line Detroit B. A) RT-PCR panel demonstrating hepatic gene expression in Detroit B derived smHLCs. B) Expression of albumin, HNF4α and A1AT at protocol endpoint, imaged using fluorescent microscopy. Scale bars=100 μm. C) Expression of ALB, A1AT (SERPINA1) and HNF4A at endpoint of protocol, as measured by TaqMan RT-qPCR. Normalised to β-actin and undifferentiated control. D) Serum protein secretion at endpoint of small molecule based protocol.

FIG. 13 shows characterization of smHLC differentiation of hiPSC line Detroit C. A) RT-PCR panel demonstrating hepatic gene expression in Detroit C derived smHLCs. B) Expression of albumin, HNF4α and A1AT at protocol endpoint, imaged using fluorescent microscopy. Scale bars=100 μm. C) Expression of ALB, A1AT (SERPINA1) and HNF4A at endpoint of protocol, as measured by TaqMan RT-qPCR. Normalised to β-actin and undifferentiated control. D) Serum protein secretion at endpoint of small molecule based protocol.

FIG. 14 shows characterization of smHLC differentiation of hESC line 360. A) Morphology at protocol endpoint, taken using phase contrast microscopy at 20×. Scale bars=100 μm. B) Expression of albumin, HNF4α and A1AT at protocol endpoint, imaged using fluorescent microscopy. Scale bars=100 μm. C) Serum protein secretion at endpoint of small molecule based protocol.

FIG. 15 shows H1 smHLCs assessed for indocyanine green uptake.

FIG. 16 shows characterization of Phase I (definitive endoderm) differentiation. A) Gene expression changes during Phase I of differentiation as measured by TaqMan RT-qPCR. B) Comparison of morphology of growth factor and small molecule definitive endoderm differentiation at days 0, 1 and 3 for growth factor, and days 0, 1 and 2 for small molecule, taken using phase contrast microscopy (10×). Scale bars=100 μm. C) Expression of FOXA2 and SOX17 at Phase I endpoint after treatment with activin/Wnt3a. CHIR99021, BIO, or Wnt3a alone, imaged using fluorescent microscopy. D) Efficiency of Phase I differentiation, determined by counting FOXA2 positive cells and SOX17 positive cells.

FIG. 17 shows characterization of Phase II (hepatic specification) differentiation A) Expression of AFP and HNF4A at Phase II endpoint of growth factor and small molecule treated cells, imaged using fluorescent microscopy. B) Efficiency of Phase II differentiation, determined by counting AFP and HNF4A double positive cells. C) Morphology at Phase II endpoint, photographed using phase contrast microscopy at 10× and 20×. Scale bars=100 μm. D) Expression of AFP CEBPA, FOXA2, GATA4, HHEX, HNF4A, PROX1, SOX17, TBX3, and TTR at Phase II endpoint following either growth factor or small molecule treatments, as measured by TaqMan.

FIG. 18 shows phase II 5 day timecourse.

FIG. 19 shows characterization of Phase III (hepatocyte like cells) differentiation: Morphology and immunofluorescence. A) Morphology of growth factor and small molecule protocol endpoints, taken using phase contrast microscopy at 10×. Scale bars=100 μm. B) Expression of albumin and HNF4A at protocol endpoints, imaged using fluorescent microscopy. C) Expression of A1AT at protocol endpoints, imaged using fluorescent microscopy. D) Expression of AFP at protocol endpoint was imaged using fluorescent microscopy. E) Efficiency of Phase III differentiation, determined by counting albumin and HNF4A double positive cells, A1AT positive cells, and AFP positive cells. F) RT-PCR of CYP7A1 gene expression of H1 derived smHLCs.

FIG. 20 shows characterization of Phase III (hepatocyte like cells) differentiation: RT-qPCR and functional analysis. A) Expression of A1AT (SERPINA1), AFP, ALB, APOA2, ASGR1, CYP3A4, HNF4A, TDO2 and TTR at endpoint of small molecule and growth factor protocols, as well as primary adult and fetal hepatocyte controls, assessed by TaqMan. B). Cytochrome P450 1A2 and 3A4 activity and induction was assessed in both smHLCs (SM) and growth factor derived HLCs (GF). C) Serum protein secretion at endpoint of both growth factor and small molecule based protocols. D) Glycogen storage in growth factor and small molecule differentiated cells as indicated by periodic acid—Schiff staining. E) H1 smHLCs treated for 1 hour with 1 mg/ml indocyanine green demonstrate uptake as assessed by phase microscopy. Scale bars=100 μm.

FIG. 21 shows hiPSC Characterisation and differentiation scheme. A) Expression of OCT4, SOX2, and NANOG, imaged using fluorescent microscopy. B) Gene expression for key pluripotency genes OCT4 (POU5F1), SOX2, and NANOG by RT-qPCR, normalised to H1 undifferentiated control. C) Schematic of the small molecule differentiation process on other pluripotent stem cell lines showing optimisation of CHIR99021 concentrations and base media compositions (+/−INS). D) Representative images of morphology throughout the differentiation procedure on Detroit hiPSC lines.

FIG. 22 shows characterization of Phase I (definitive endoderm) differentiation of multiple pluripotent lines. A) Gene expression changes during Phase I of differentiation assessed by RT-qPCR. B) Typical morphology of small molecule derived definitive endoderm end points taken using phase contrast microscopy (10×). C) Expression of FOXA2 and SOX17 at Phase I endpoint after treatment with CHIR99021 imaged using fluorescent microscopy. D) Efficiency of Phase I differentiation for each line was determined by counting FOXA2 positive cells and SOX17 positive cells.

FIG. 23 shows a phase I 48 hour time course to assess transcriptional developmental trajectory.

FIG. 24 shows characterization of Phase II (hepatic specification) on multiple pluripotent lines. A) Typical morphology observed at Phase II endpoint, photographed using phase contrast microscopy at 10×. B) Expression of AFP and HNF4A at Phase II endpoint of small molecule treated cells, imaged using fluorescent microscopy. C) Efficiency of Phase II differentiation was determined for each line by counting HNF4A and AFP double positive cells. D) Expression of AFP, CEBPA, FOXA2, GATA4, HHEX, HNF4A, PROX1, SOX17, TBX3, and TTR at Phase II endpoint, measured by TaqMan.

FIG. 25 shows a phase II 5 day time course.

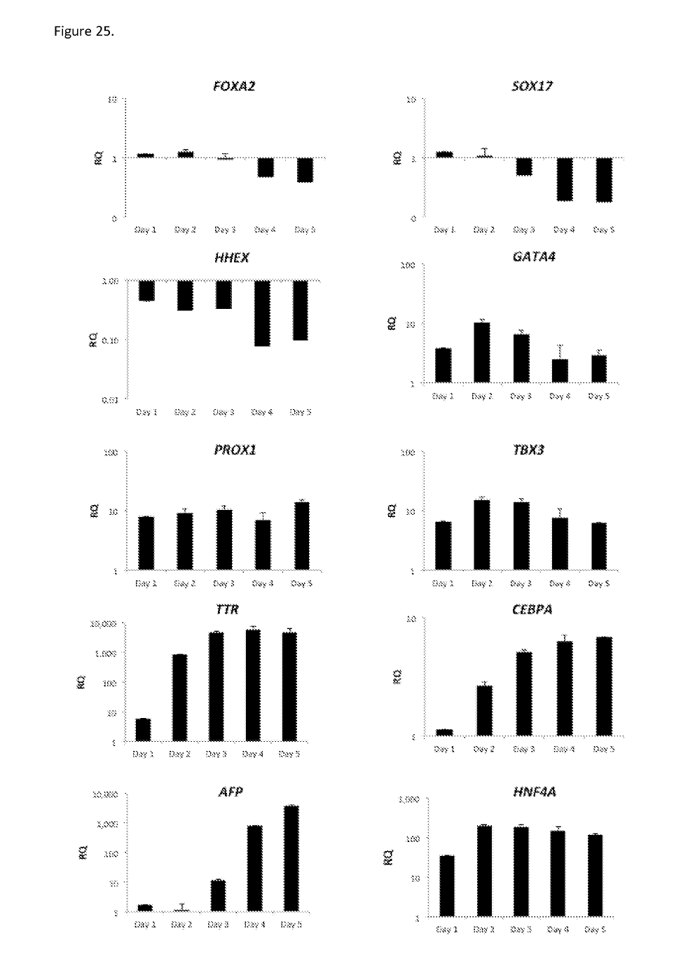

FIG. 26 shows characterization of Phase III (hepatocyte like cells) differentiation: Morphology and immunofluorescence. A) Morphology of hESC line 207, hiPSC lines Detroit RA, RB and RC at small molecule protocol endpoint (day 17), taken using phase contrast microscopy at 10×. B) Expression of albumin and HNF4A at protocol endpoint imaged using fluorescent microscopy. C) Expression of alpha-1-antitrypsin at protocol endpoints, imaged using fluorescent microscopy.

FIG. 27 shows characterization of Phase III (hepatocyte like cells) RT-qPCR and functional analysis. A) Serum protein secretion at endpoint of small molecule protocol for all lines. B) Assessment of RT-PCR of CYP7A1 gene expression of hiPSC derived smHLCs. Lane 1=hESC H1 control, lane 2=Detroit RA, Lane 3=Detroit RB, Lane 4=Detroit RC day 17 smHLCs CYP7A1 expression, lower panel=ACTB loading control. C) Efficiency of Phase III differentiation, determined by counting albumin and HNF4A double positive cells and A1AT positive cells. D) Expression of A1AT (SERPINA1), AFP, ALB, APOA2, ASGR1, CYP3A4, HNF4A, TDO2 and TTR at endpoint of small molecule protocol, as well as growth factor derived HLCs, primary adult and fetal hepatocyte controls, as measured by TaqMan.

### DEFINITIONS

To facilitate an understanding of the present invention, a number of terms and phrases are defined below:

As used herein, the terms “detect”, “detecting” or “detection” may describe either the general act of discovering or discerning or the specific observation of a detectably labeled composition.

As used herein, the term “subject” refers to any organisms that are investigated or screened using the methods described herein. Such organisms preferably include, but are not limited to, mammals (e.g., murines, simians, equines, bovines, porcines, canines, felines, and the like), and most preferably includes humans.

The term “diagnosed,” as used herein, refers to the recognition of a disease by its signs and symptoms, or genetic analysis, pathological analysis, histological analysis, and the like.

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “DMSO mimetic” refers to a reagent (e.g., molecule or compound) that has one or more biological functions of DMSO (e.g., driving differentiation of pluripotent cells). In some embodiments, the DMSO mimetic is a polar aprotic solvent (e.g., a polar organosulfur solvent).

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Such examples are not however to be construed as limiting the sample types applicable to the present invention.

As used herein the term “stem cell” (“SC”) refers to cells that can self-renew and differentiate into multiple lineages. A stem cell is a developmentally pluripotent or multipotent cell. A stem cell can divide to produce two daughter stem cells, or one daughter stem cell and one progenitor (“transit”) cell, which then proliferates into the tissue's mature, fully formed cells. Stem cells may be derived, for example, from embryonic sources (“embryonic stem cells”) or derived from adult sources. For example, U.S. Pat. No. 5,843,780 to Thompson describes the production of stem cell lines from human embryos. PCT publications WO 00/52145 and WO 01/00650 describe the use of cells from adult humans in a nuclear transfer procedure to produce stem cell lines.

Examples of adult stem cells include, but are not limited to, hematopoietic stem cells, neural stem cells, mesenchymal stem cells, and bone marrow stromal cells. These stem cells have demonstrated the ability to differentiate into a variety of cell types including adipocytes, chondrocytes, osteocytes, myocytes, bone marrow stromal cells, and thymic stroma (mesenchymal stem cells); hepatocytes, vascular cells, and muscle cells (hematopoietic stem cells); myocytes, hepatocytes, and glial cells (bone marrow stromal cells) and, indeed, cells from all three germ layers (adult neural stem cells).

As used herein, the term “embryonic stem cell” (“ES cell” or ESC”) refers to a pluripotent cell that is derived from the inner cell mass of a blastocyst (e.g., a 4- to 5-day-old human embryo), and has the ability to yield many or all of the cell types present in a mature animal

As used herein, the term “induced pluripotent stem cells” (“iPSCs”) refers to a stem cell induced from a somatic cell, e.g., a differentiated somatic cell, and that has a higher potency than said somatic cell. iPS cells are capable of self-renewal and differentiation into mature cells.

As used herein, the term “totipotent cell” refers to a cell that is able to form a complete embryo (e.g., a blastocyst).

As used herein, the term “pluripotent cell” or “pluripotent stem cell” refers to a cell that has complete differentiation versatility, e.g., the capacity to grow into any of the mammalian body's approximately 260 cell types. A pluripotent cell can be self-renewing, and can remain dormant or quiescent within a tissue. Unlike a totipotent cell (e.g., a fertilized, diploid egg cell), a pluripotent cell, even a pluripotent embryonic stem cell, cannot usually form a new blastocyst.

As used herein, the term “multipotent cell” refers to a cell that has the capacity to grow into a subset of the mammalian body's approximately 260 cell types. Unlike a pluripotent cell, a multipotent cell does not have the capacity to form all of the cell types.

As used herein, the term “progenitor cell” refers to a cell that is committed to differentiate into a specific type of cell or to form a specific type of tissue.

As used herein the term “feeder cells” refers to cells used as a growth support in some tissue culture systems. Feeder cells may be embryonic striatum cells or stromal cells.

### DETAILED DESCRIPTION OF THE INVENTION

The present invention relates to compositions and method for differentiating stem cells. In particular, the present invention relates to methods of generating differentiated cells (e.g., hepatocytes) from human pluripotent stem cells (hPSCs) using a small molecule-driven approach

Experiments conducted during the development of the present invention resulted in a method of generating hepatocytes from human pluripotent stem cells (hPSCs) using a small molecule-driven approach, which does not require the use of growth factors to direct differentiation. The method is assembled into 3 phases: (i) Definitive endoderm (DE) specification, (ii) Hepatic endoderm specification and (iii) Mature hepatocyte production. In order to generate the cell types of each phase the cells should pass through developmentally relevant stages such as primitive streak and mesendoderm, as adjudged by gene transcription and protein expression.

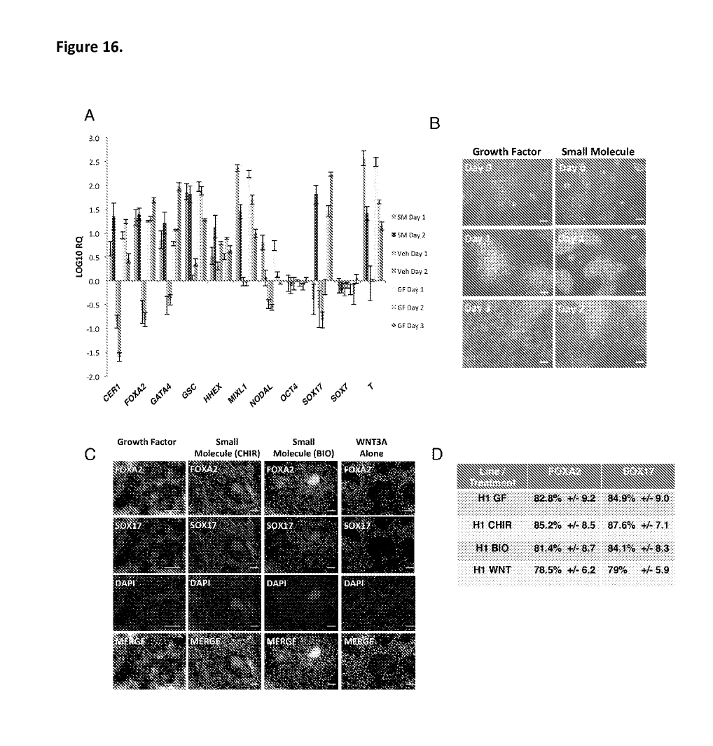

Described herein is the development of a protocol for the efficient differentiation of functional HLCs from hPSCs, which does not require the addition of recombinant growth factors and is applicable to both hESCs and hiPSCs. During the development of the protocol, it came as a surprise to discover that pluripotent cells can be differentiated to DE with a short pulse (24 hrs) of either CHIR99021, BIO, or Wnt3a without the inclusion of activin A. The rationale to examine GSK-3 inhibition as a route to producing DE has a basis in the observations of Lickert and colleagues (Engert et al., Development 140, 3128-3138 2013) who demonstrated that Wnt signalling regulates SOX17 expression. The results clearly demonstrate that GSK-3 inhibition alone is sufficient to produce DE, a finding, which in itself represents an opportunity for major cost saving in hepatocyte production. Importantly, this first stage provided productive DE, which expressed a battery of DE markers including SOX17 and FOXA2, and passed through the equivalent developmental points as growth factor DE (FIG. 16A). The use of was shown to be successful when employed following CHIR99021 treatment as with activin A and Wnt3a. A rapid change in morphology, coupled with the appearance of the hepatic progenitor markers HNF4A and AFP, both at the transcriptional and protein level was observed. The final phase of this protocol was to take hepatic progenitors and provide the environment for hepatocyte maturation in order to generate HLCs. A combination of two small molecules of the glucocorticoid family, DEX and hydrocortisone-21-hemisuccinate (HC) were used along with a potent HGF mimetic called N-hexanoic-Tyr, Ile-(6) aminohexanoic amide or Dihexa in Phase III of the protocol. L-15 media was supplemented with the aforementioned small molecules and shown to efficiently generate HLCs from both hESCs and hiPSCS, which displayed typical hepatic morphology and expressed a number of hepatic markers at the transcriptional and protein level. The smHLCs exhibited key functional attributes including serum protein production and cytochrome P450 metabolism. The methods described herein provides a means to cut the costs associated with hepatocyte production. A combination of this small molecule approach with the steadily advancing field of cell culture automation allows for the production of high quality hepatocytes from stem cells at large scale for industrial and clinical translation.

In some embodiments, the differentiation method is divided into three phases that are performed sequentially. In phase 1, pluripotent cells are contacted with a GSK-3 inhibitor. Examples of GSK inhibitors include, but are not limited to, metal cations (e.g., beryllium, copper, lithium, mercury, or tungsten), dibromocantharelline, hymenialdesine, indirubins, meridianins, aminopyrimidines (e.g., CT98014, CT98023, CT99021, or TWS119), CHIR99021, arylindolemaleimides (e.g., SB-216763 or SB-41528), thiazoles (e.g., AR-A014418 or AZD-1080), paullones (e.g., alsterpaullone, cazpaullone or kenpaullone), aloisines (IC50=0.5-1.5 μM), manzamine A, paliurine, tricantine, thiadiazolidindiones (e.g., TDZD-8, NP00111, NP031115, or tideglusib), halomethylketones (e.g., HMK-32), BIO, Wnt3a, or peptides (e.g., L803-mts). In some embodiments, the GSK inhibitor is CHIR99021, BIO and Wnt3a.

In some embodiments, cells are next contacted with DMSO or a DMSO mimetic (Phase II).

In some embodiments, phase III comprises a glucocorticoid (e.g., dexamethasone (DEX) or hydrocortisone 21-hemisuccinate) and/or HGF mimic. HGF mimetic include, for example, an angiotensin IV analog derivative. Examples include, but are not limited to, Nle1-AngIV; N-Acetyl-Nle-Tyr-Ile-His; D-Nle-Tyr-Ile; GABA-Tyr-Ile; Nle-Tyr-Ile-His-NH2; D-Nle-X-Ile-NH—(CH2)5-CONH2, where X is any amino acid, preferably Try, Cys, Trp, Asp, Phe, or Ser; Nle1-AngIV (Nle1-Tyr2-Ile3-His4-Pro5-Phe6) and its 1-3 aa C-terminal truncations: Nle1-Tyr2-Ile3-His4-Pro5, Nle1-Tyr2-Ile3-His4, and Nle1-Tyr2-Ile3 (See e.g., McCoy J Pharmacol Exp Ther 344:141-154 (2013); Kawas, the Journal of Pharmacology and Experimental Therapeutics Vol. 340, No. 3 p. 539 (2012); and Benoist, the Journal of Pharmacology and Experimental Therapeutics Vol. 339, No. 1 p. 35 (2011); each of which is herein incorporated by reference in its entirety).

In some embodiments, media described herein (e.g., in the experimental section) are utilized. However, any suitable basic growth medium for hepatocytes is contemplated.

In some embodiments, markers for differentiation are assessed after each phase. Exemplary markers for differentiation into hepatocytes include, but are not limited to, Phase 1: FOXA2, SOX17, CER1, HHEX, GSC, MIXL1, CXCR4; Phase 2: AFP, HNF4A, FOXA2; and Phase 3: ALB, HNF4A, A1AT (SERPINA), ASGPR1/2, Fibronectin, Fibrinogen, Transtherytin (TTR), CYP450s (1A2, 2C9/19, 2D6, 3A4), Uridine 5′-diphospho-glucuronosyltransferases (UGTs), glutathione S-transferase (GSTs), Drug transporters.

The present disclosure is not limited to particular pluripotent stem cells to be used in differentiation. In some embodiments, embryonic (hESC) or induced pluripotent stem cells (hiPSC) derived from somatic cells (e.g. fibroblasts) are utilized.

Provided herein are multistep cell culture procedures for differentiating stem cells into cell populations comprising hepatic cells. In some embodiments, cells are placed in a series of culture conditions (e.g., multiple stages), for proscribed time periods. In some embodiments, culture media is changed regularly (e.g., hourly, four-times daily, twice daily, daily, etc.). In some embodiments, culture media is continuously replenished. In some embodiments, cell culture is carried out at room temperature (e.g., 18° C., 19° C., 20° C., 21° C., 22° C., 23° C., 24° C., or ranges therein). In some embodiments, reagents used in culture media are sterilized. In some embodiments, devices (e.g., chambers, vessels, bottles, flasks, tubes, etc.) used in culturing are sterilized.

In some embodiments, appropriate devices are selected for containing cells and media during the various stages of culturing (e.g., Transwell) and/or post-culturing shipping (e.g., conical tube, etc.), clustering (etc., AggreWell), expansion (e.g., Spinner flask, etc.), etc. One commercially available device that finds use in some embodiments described herein are Transwell cell culture chambers, or Transwell plates (e.g., available from Costar Corp., Cambridge, Md., USA). Each chamber of a Transwell plate comprises a flat-bottomed, open-topped, lower compartment with impermeable bottom and sides, and an open-topped, upper compartment with a microporous membrane which forms the bottom of the upper compartment. This assembly is typically covered by a removable lid. In use, cells are placed on the upper surface of the microporous membrane within the upper compartment. The upper compartment is inserted into the lower compartment. Due to the permeability of the membrane, media, nutrients, factors, etc. are able to traverse the membrane.

The protocol described herein in exemplified for the differentiation of pluripotent cells into hepatocytes. However, the systems and method described herein find use in the differentiation of pluripotent cells into a variety of cell types. Examples include, but are not limited to, cells of endodermal lineage (e.g., pancreas (e.g. insulin producing cells), lung, colon, and intestinal cell types (foregut & hindgut), and liver).

For example, in some embodiments, the cells are hepatocytes. Hepatocytes are a cell of the main tissue of the liver. Hepatocytes make up 70-85% of the liver's cytoplasmic mass. These cells are involved in protein synthesis, protein storage, transformation of carbohydrates, synthesis of cholesterol, bile salts and phospholipids, detoxification, modification, and excretion of exogenous and endogenous substances, and formation and secretion of bile.

In some embodiments, cells are differentiated into pancreatic (e.g. insulin producing cells). Beta cells (beta-cells, β-cells) are a type of cell in the pancreas located in the islets of Langerhans. They make up 65-80% of the cells in the islets and are responsible for producing, storing and releasing insulin.

In some embodiments, the differentiated cells are cholangiocytes. Cholangiocytes are the epithelial cells of the bile duct and contribute to bile secretion via net release of bicarbonate and water.

In some embodiments, differentiated cells are intestinal cells. In some embodiments, intestinal cells are epithelial cells or enterocytes (intestinal absorptive cells). Enterocytes are involved in uptake of nutrients and secretion of immunoglobulins.

Additional examples of differentiated cells include, but are not limited to, trichocyte, keratinocyte, gonadotrope, corticotrope, thyrotrope, somatotrope, lactotroph, neuron, glia (Schwann cell), satellite glial cell, chromaffin cell, parafollicular cell, glomus cell, melanocyte, nevus cell, merkel cell, odontoblast, cementoblast, corneal keratocyte, oligodendrocyte astrocyte, ependymocytes, pinealocyte, pneumocyte, clara cell, goblet cell, G cell, D cell, ECL cell, gastric chief cell, parietal cell, foveolar cell, K cell, S cell, D cell, I cell, paneth cell, microfold cell, hepatocyte, hepatic stellate cell, cholecystocyte, centroacinar cell, pancreatic stellate cell, alpha cell, beta cell, delta cell, F cell, epsilon cell, follicular cell, parathyroid chief cell, oxyphil cell, urothelial cells, osteoblast, osteocyte, chondroblast, chondrocyte, myofibroblast, fibroblast, fibrocyte, myoblast, myocyte, myosatellite cell, tendon cell, cardiac muscle cell, lipoblast, adipocyte, red blood cells, white blood cells, interstitial cell of Cajal, angioblast, endothelial cell, mesangial cell, juxtaglomerular cell, macula densa cell, stromal cell, interstitial cell, telocytes, simple epithelial cell, podocyte, sertoli cell, leydig cell, granulosa cell, peg cell, spermatozoon, ovum, lymphocytes, myeloid cells, angioblast/mesoangioblast, pericyte, Mural cell, etc.

The differentiated cells described herein find use in a variety of research, screening, clinical, and therapeutic applications. For example, in some embodiments, hepatocytes are utilized for drug screening and development (e.g., drug toxicity screening, assessing drug metabolism, and toxicity (e.g., using genetically defined lines for personalized therapy)).

In some embodiments, hepatocytes find use in clinical and therapeutic applications (e.g., creating bioartifical liver devices, cell therapy (e.g., engraftment of artificially derived cells), and clinical grade cell production for therapeutics. In some embodiments, hepatocytes find use in disease modeling (e.g., use of derived cells for engraftment in animal models) and drug discovery and basic research. The present disclosure is not limited to the uses described herein. Additional uses are specifically contemplated.

In some embodiments, hepatocytes or other cells are differentiated in three-dimensional scaffolds (e.g., for cell or tissue therapy or screening applications).

### EXPERIMENTAL

The following examples are provided in order to demonstrate and further illustrate certain preferred embodiments and aspects of the present invention and are not to be construed as limiting the scope thereof.

### Example 1

Materials and Methods

Cell Culture.

H1 hESCs (WiCell), 207 and 360 hESCs (Ström S, et al., In Vitro Cell Dev Biol Anim. 2010; 46:337-44) and Detroit 551 (ATCC CCL-110) hiPSCs were maintained at 37° C./5% CO₂ in feeder-free conditions using Growth Factor Reduced Matrigel (BD Biosciences) and E8 Medium (Life Technologies), with routine passaging performed at a 1:3 ratio using 0.5 mM ethylenediaminetetraacetic acid (EDTA) (Life Technologies). All Matrigel plates were coated with a 1:48 dilution in Advanced DMEM-F12 (Life Technologies) and incubated at 37° C./5% CO₂ for 1 hour prior to use.

Hepatocyte Differentiation.

Briefly, hPSCs were differentiated via a three phase protocol as follows: Phase I consisted of DE induction through the use of the small molecule CHIR99021; Phase II induced the specification of DE into hepatic progenitor cells via the use of DMSO; and in Phase III the hepatic progenitors matured into HLCs through the combined use of dexamethasone, hydrocortisone 21-hemisuccinate, and the HGF mimetic Dihexa.

Reagents were obtained from commercial sources as listed below.

Essential 8 Medium (Essential 8 Basal Medium, Life Technologies, cat. no. A15169-01; Essential 8 Supplement (50×), Life Technologies, cat. no. A15171-01)
EDTA
ECM Gel, Growth Factor Reduced, without Phenol Red from Engelbreth-Holm-Swarm mouse sarcoma (Sigma-Aldrich) Cat. no. E6909
Geltrex LDEV-Free hESC-qualified Reduced Growth Factor Basement Membrane Matrix without phenol red (Gibco) A1413302
RPMI Medium 1640+GlutaMAX (Life Technologies) Cat no. 61870
B-27 Supplement with Insulin (Life Technologies) Cat no. 17504-044
B-27 Supplement without insulin (Life Technologies) Cat no. 05-0129SA
Stemolecule™ CHIR 99021 (Stemgent) Cat no. 04-0004
BIO (2′Z,3′E)-6-Bromoindirubin-3′-oxime (Tocris) Cat no. 3194
Wnt3A recombinant mouse (Peprotech) Cat no. 315-20
DPBS (1×) [—]CaCl2 [−]MgCl2 (Life Technologies) Cat no. 14190
Knockout DMEM (Life Technologies) Cat no. 10829
Knockout Serum Replacement (Life Technologies) Cat no. 10828
MEM Non-essential Amino Acids (Life Technologies) Cat no. 11140-035
Cell Therapy Systems GlutaMAX-1 CTS (100×) (Life Technologies) Cat no. A12860-01
Dimethylsulfoxide (DMSO) (Sigma-Aldrich) Cat no. 472301-100 mL
2-Mercaptoethanol (Life Technologies) Cat no. 31350-010
L-15 Medium Leibovitz (Sigma-Aldrich) L5520-500 mL
Fetal Bovine Serum (South American Origin). Sterile Filtered. (Biowest) Cat no. 51800-500
Tryptose Phosphate Broth Solution (Sigma Aldrich) T8159-100 mL
Insulin-Transferrin-Selenium (Life Technologies) Cat no. 41400-045
(+)-Sodium L-ascorbate (Sigma Aldrich) A7631-25G
HC21-hydrocortisone 21-hemisuccinate (Sigma-Aldrich) H4881
Dihexa (Gift Joseph Harding University of Washington)
Dexamethasone (Sigma Aldrich) D1756.

RNA Isolation, RT-qPCR, Immunofluorescence, Protein Analysis, and Hepatic Function Analysis.

These analyses were performed in line with previously established techniques and according to manufacturer's instructions in the case of ready-to-use kits.

### Results

Production of Definitive Endoderm (DE) Using GSK-3 Inhibition (Phase I)

The ability to produce hepatocytes from hPSCs that have utility in both clinical and research arenas is benefited by methodologies that are robust in terms of efficiency and reproducibility. The majority of methodologies to date are far from this and also reliant on recombinant growth factors to direct cellular fate. This will prove a major hurdle if these cells are to be utilised in a therapeutic environment. A differentiation procedure that is devoid of growth factors and driven by small molecules was developed. The procedure is notionally trisected into 3 phases inducing DE differentiation (Phase I), hepatic specification (Phase II) and hepatocyte maturation (Phase III).

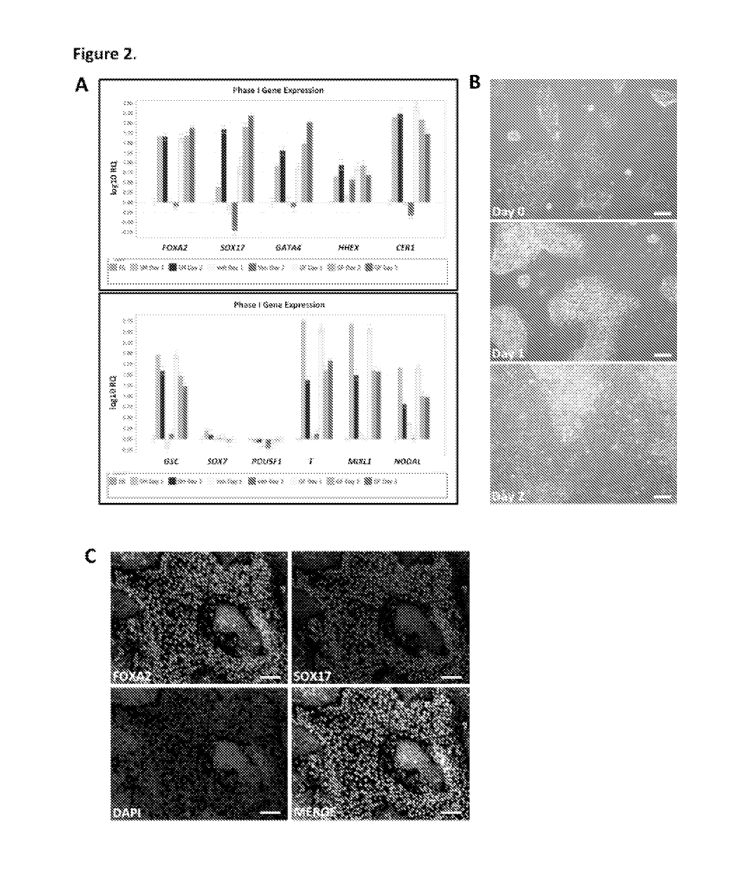

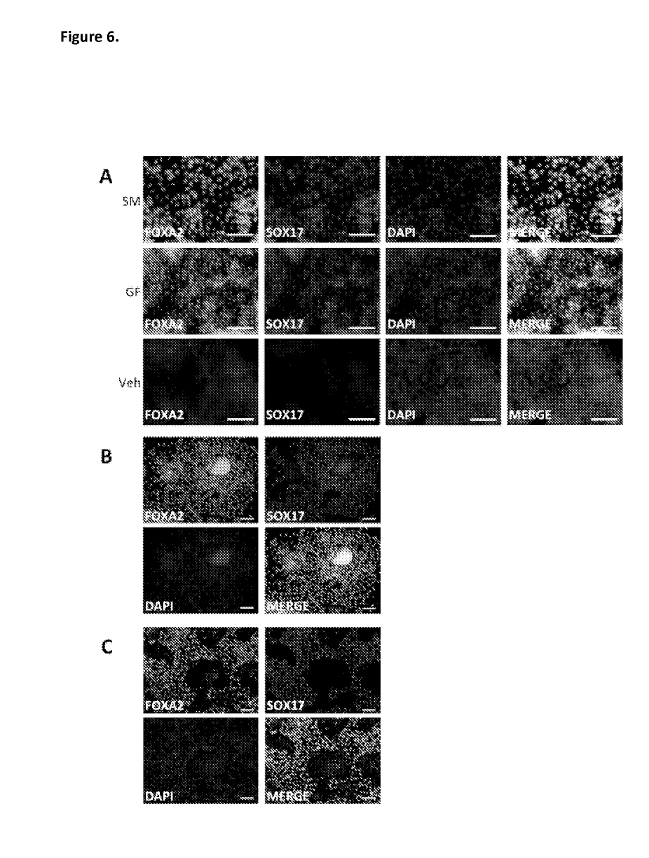

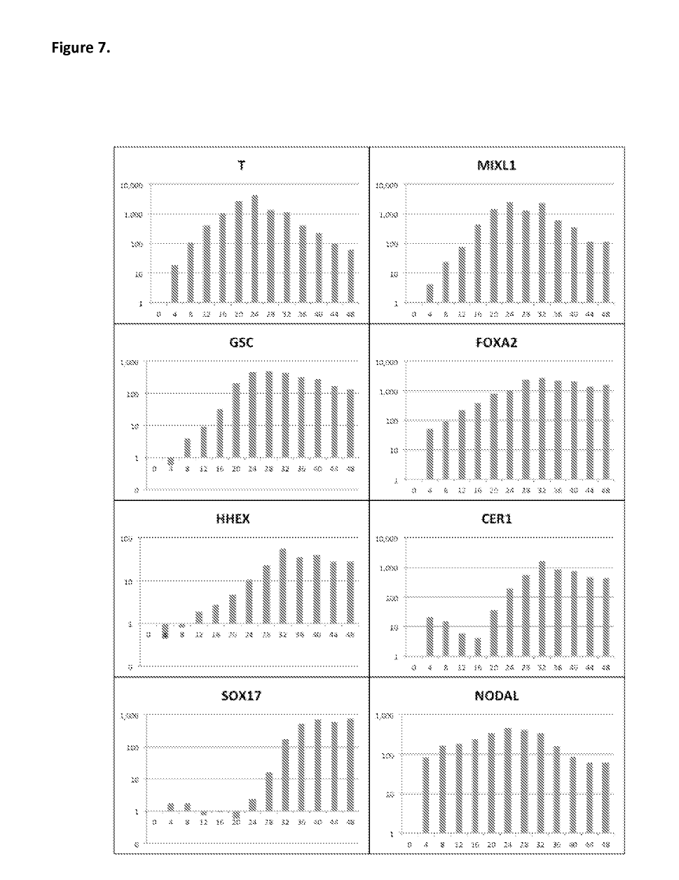

Following studies of the utility of GSK-3 inhibition in priming pluripotent cells for endodermal differentiation (Tahamtani, Y., et al., Stem Cells Dev, 2013, 22(9): p. 1419-32), and reports that Wnt/β-Catenin signalling regulates SOX17 expression and is essential for endoderm formation (Engbert S, et al., Development 2013; 140:3128-38), it was established whether this approach was suitable as a starting point for the generation of functional hepatocytes. The conditions for DE differentiation were established in the hESC line H1. Through a 24 hour treatment with 3 μM CHIR99021, followed by 24 hours of non-directed differentiation in RPMI-B27, it was possible to guide hPSCs through developmentally relevant stages to produce a population of definitive endoderm (FIG. 2a). Over a 48 hour period dynamic changes in the gene expression pattern were observed (FIG. 7). By 48 hours, elevated expression of DE markers such as FOXA2, GSC, SOX17, HHEX and CER1 was observed (Sasaki H and Hogan B L M. Development 1993; 118:47-59; Ang S L, et al., Development 1993; 119, 1301-1315; Monaghan A P, et al., Development 1993; 119:567-78; Blum M, et al., Cell. 1992; 26:1097-106; Kanai-Azuma M, et al., Development. 2002; 129:2367-79). In addition, early events of the differentiation indicated transition through a primitive streak (PS) intermediate. A rapid upregulation of NODAL was observed within 4 hours of exposure to CHIR99021, which is indicative of a transition towards a PS population (FIG. 7) (Lu C C, et al., Curr Opin Genet Dev 2001; 11:384-92). This is followed by induction of the PS markers T and GSC (FIG. 7). The markers SOX17, GSC, FOXA2 and MIXL1 are expressed in extra-embryonic endodermal lineages as well as definitive endoderm. In order to demonstrate that the differentiation procedure was not producing primitive endoderm, the levels of SOX7 were examined. No upregulation was observed during the procedure (FIG. 2A). The observed patterns of expression are similar to those seen with a 3 day treatment of activin A and Wnt3a or 5 day treatment of activin A (D'Amour K A, et al., Nat Biotechnol. 2005; 23:1534-41). These changes in gene expression were accompanied by morphological changes; the cells shifted from a hESC morphology to dense, bright clusters at 24 hours, followed by a petal like morphology at 48 hours (FIG. 2B). At the 48 hour timepoint (Phase I endpoint), co-expression of FOXA2 and SOX17 were observed at the protein level using immunofluorescence (FIG. 2C). Treatments with activin A/Wnt3a, CHIR99021 and vehicle control were compared and equivalent co-expression of the DE proteins FOXA2 and SOX17 was observed by immunofluorescence in the growth factor and small molecule treated approaches, and no co-expression in the control (FIG. 6A). Next it was assessed if GSK-3 inhibition was a generic mechanism to drive hPSCs to DE. FIG. 6B demonstrates the utility of an alternative GSK-3 inhibitor (BIO—1 μM) to produce FOXA2/SOX17 positive cells under the same conditions, indicating that GSK-3 inhibition followed by its removal is responsible for commitment to DE, BIO and CHIR99021 are potent pharmacological GSK-3 specific inhibitors that result in the activation of the Wnt signalling pathway (Sato N, et al., Nature Medicine 2004; 10: 55-63; Sineva G S and Pospelov V A. Biol. Cell 2010; 102:549-560), so the ability of the protein Wnt3a alone to drive differentiation towards DE was assayed. FIG. 6C demonstrates that treatment with Wnt3a was sufficient to produce populations of cells that expressed the DE markers FOXA2 and SOX17. This observation indicates that Wnt3a treatment alone can facilitate the production of DE, and that the inclusion of activin A is not necessary for definitive endoderm production in vitro.

Hepatic Specification Through DMSO Treatment of Definitive Endoderm (Phase II).

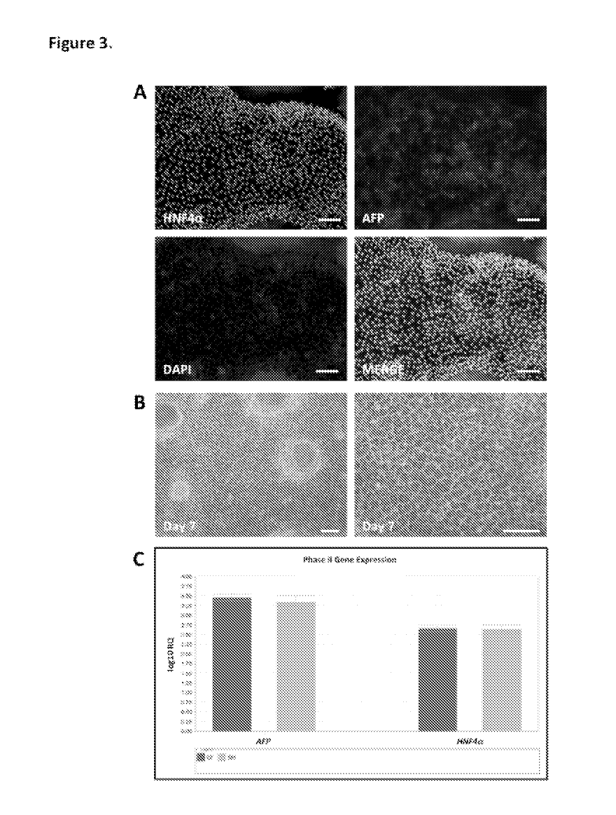

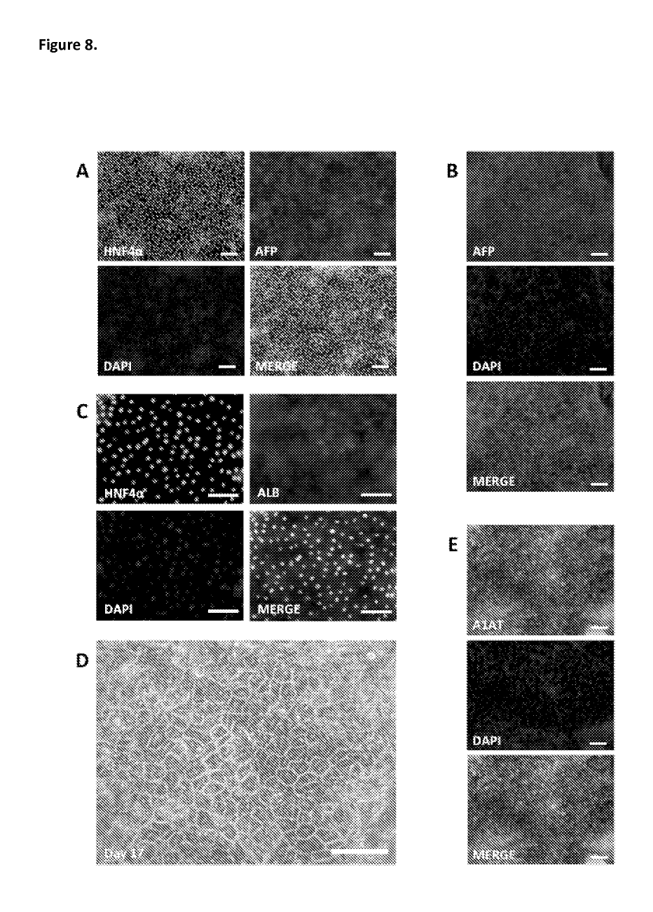

Following the production of definitive endoderm through small molecule stimulation, the next step was to specify a hepatic fate. Routes to efficiently produce an AFP/HNF4α positive hepatic progenitor population were investigated. A 5 day treatment of 1% DMSO was utilized (Lu et al., supra; Sullivan et al., supra; Rambhatla L, et al., Cell Transplant 2003; 12:1-11; Soto-Gutierrez A, et al. Cell Transplant. 2006; 15:335-41). On subjecting DE to Phase II conditions (DMSO) a rapid change in morphology and a spurt of proliferation was observed. After the 5 days of treatment greater than 90% co-expression of AFP and HNF4α, as assessed by immunofluorescence, was observed (FIG. 3A). In addition the cells exhibited typical hepatocyte progenitor morphology as assessed by phase contrast microscopy (FIG. 3B). The levels of AFP/HNF44α co-expression observed are indicative that this phase of the differentiation is extremely efficient. The expression levels of HNF4A and AFP in cells derived from the small molecule approach were compared with cells differentiated via standard in house growth factor method. FIG. 3C clearly demonstrates similar levels of expression of HNF4A and AFP at the transcriptional level. Additionally, it was demonstrated that this approach (DMSO) following activin A/Wnt3a treatment also produces HNF4α/AFP co-expression at the protein level, in line with previous reports (FIG. 8A).

Production of HLCs Via Dexamethasone and the HGF Receptor Agonist Dihexa (Phase III).

The final stage of HLC differentiation (hepatic maturation) has been performed using a wide range of growth factors such as HGF, OSM, FGF4, VEGF and EGF (Han et al., supra). A potent, stable HGF receptor agonist, N-hexanoic-Tyr, Ile-(6) aminohexanoic amide (Dihexa) was identified for use in hepatic maturation. This molecule was originally developed as a potential therapeutic intervention for neurodegenerative disorders such as Alzheimer's disease (McCoy A T, et al., J Pharmacol Exp Ther. 2013; 344:141-54.). In addition, the small molecule glucocorticoid mimetic dexamethasone (DEX), which is well established in hepatocyte maturation procedures, was used.

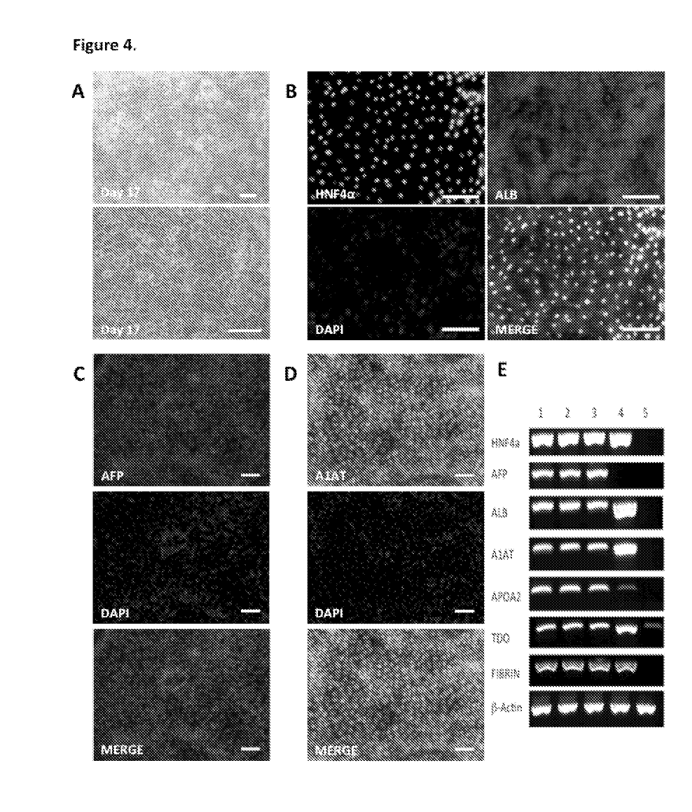

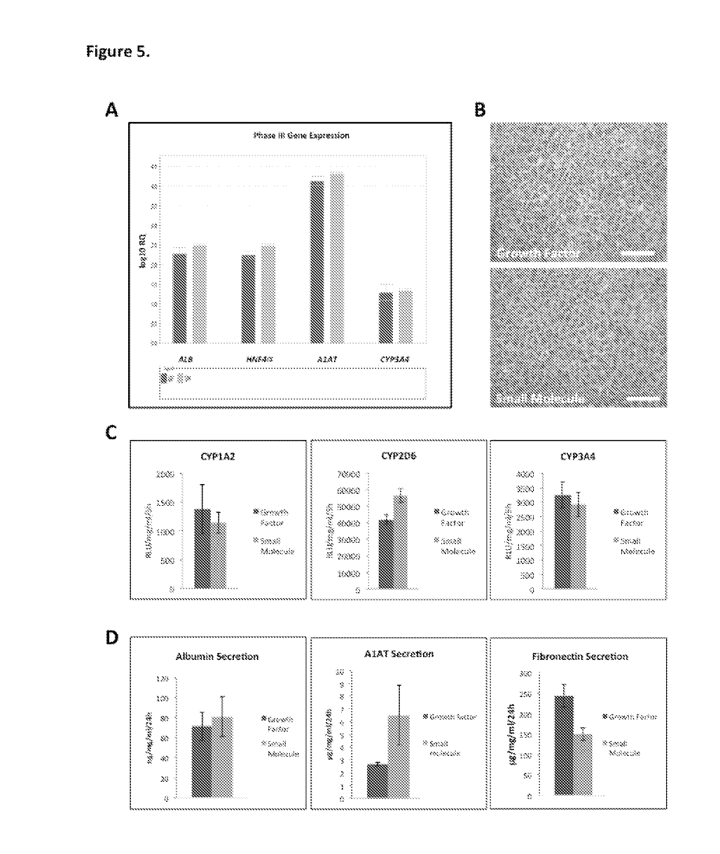

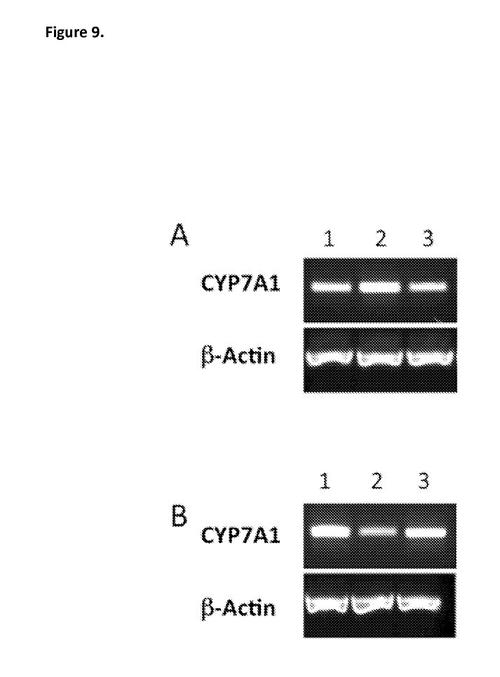

A number of base media were tested to assess the optimal concentration of DEX and Dihexa. The media HepatoZYME (Life technologies) was used to establish the optimal concentrations of DEX and Dihexa as being 100 nM for each. However, HepatoZYME contains the growth factor EGF, thus was unsuitable (Garcia M C, et al., Drug Metab Dispos. 2001; 29:111-20). Williams base media was next assessed. Both DEX and Dihexa were required and the above concentrations gave the best results in terms of morphology and function. A modified formulation of Leibovitz L-15 media (L-15), which has been described in the literature as a standard method to generate mature hepatocytes (Sullivan et al., supra; Hay et al., supra) was utilized for further experiments. L-15 media was supplemented with DEX and Dihexa (both at 100 nM), which led to the production of cells displaying typical hepatocyte morphology at the endpoint of the small molecule driven differentiation protocol (FIG. 4A). The cells were large and angular with bright junctions and in some instances contained multiple nuclei. The resulting HLCs demonstrated expression of the hepatocyte markers albumin, AFP, HNF4α and alpha-1-antitrypsin by immunofluorescence (FIGS. 4B, C and D). Comparable data can be seen for growth factor based differentiation in Supporting FIGS. 3B, C, D and E. Gene expression was analysed by RT-PCR (FIG. 4E). The panel shows a repertoire of hepatic markers that were expressed: HNF4A, AFP, ALB, A1AT, APOA2, TDO and FIBRIN. To corroborate that the source of the HLCs is from definitive endoderm rather than yolk sac, it was demonstrated that the definitive endoderm origin marker CYP7A1 was expressed (Asahina K, et al., Genes Cells. 2004; 9:1297-308) (FIG. 9A). To ensure that the protocol was equivalent to previously described growth factor based methods, functional hepatocytes were produced as described by Hay and colleagues (supra) (see FIGS. 8B, C, D and E) for validation. The levels of a number of hepatocyte markers ALB, AFP, CYP3A4 and alpha-1-antitrypsin (SERPINA1) were compared by TaqMan RT-qPCR. The observed relative levels of expression were very similar irrespective of being derived via growth factor or small molecule based protocol (FIG. 5A).

Small Molecule Derived HLCs Demonstrate Hepatic Function.

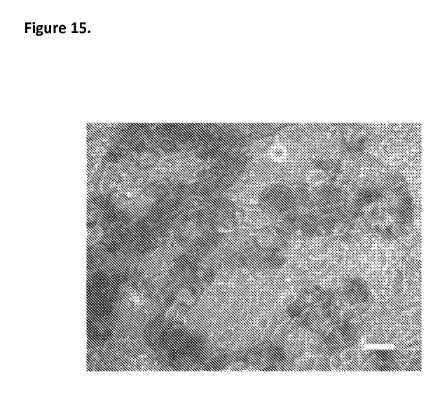

Above it was shown that small molecule derived HLCs exhibit hepatic morphology and expression of hepatocyte specific markers at the transcriptional and protein level. It was next assessed if small molecule derived HLCs (smHLCs) displayed functional hepatic characteristics. An important function of hepatocytes is the ability to clear xenobiotics via metabolism through the cytochrome P450 iso-enzymes. smHLCs were assessed for their metabolic potential, as compared to growth factor derived HLCs. The cytochrome P450 enzymes are critical in drug metabolism, in particular CYP1A2, CYP2D6 and CYP3A4. The function of these CYP450s was assessed via the generation of luminescent metabolites, similar levels of CYP450 activity to those seen in the growth factor protocol were observed (FIG. 5C). Another key function of hepatocytes is the production of serum proteins. The ability of the smHLCs to secrete albumin, alpha-1-antitrypsin and fibronectin were assessed by ELISA. All three proteins were detected in the medium at levels similar to those seen from growth factor derived cells (FIG. 5D). Another function tested was the ability to store glycogen. smHLCs were stained with periodic acid —Schiff (PAS), and the nuclei counterstained with hematoxylin and eosin. Extensive cytoplasmic staining (pink to purple), indicative of glycogen storage, at levels similar to those observed in growth factor derived hepatocytes was observed (FIG. 5B). The uptake of indocyanine green (ICG) was also observed, after a brief treatment, ICG positive cells were clearly visible (FIG. 15).

smHLCs can be Derived from Multiple Human Pluripotent Stem Cell Lines.

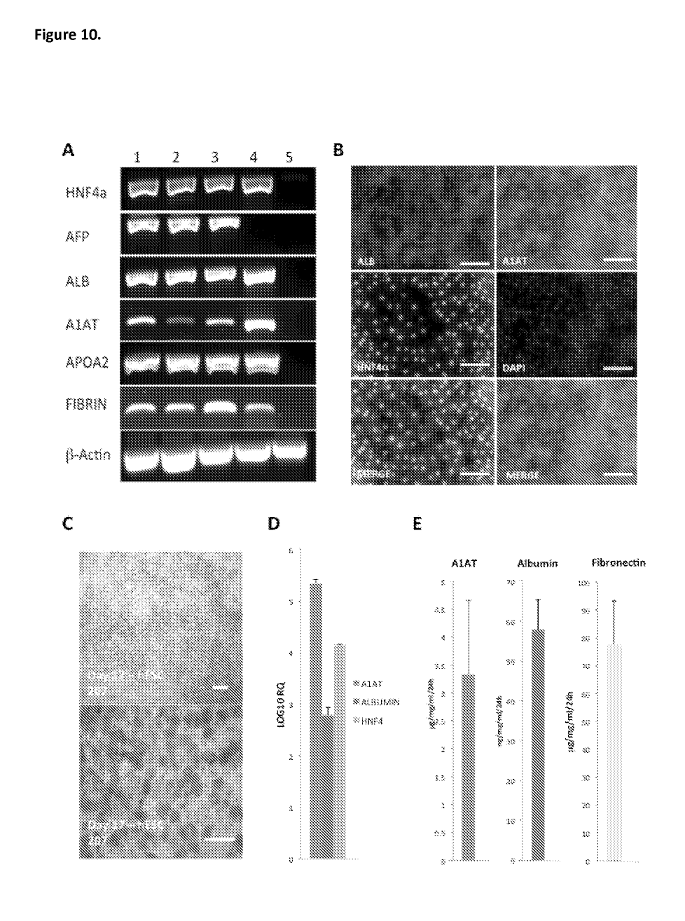

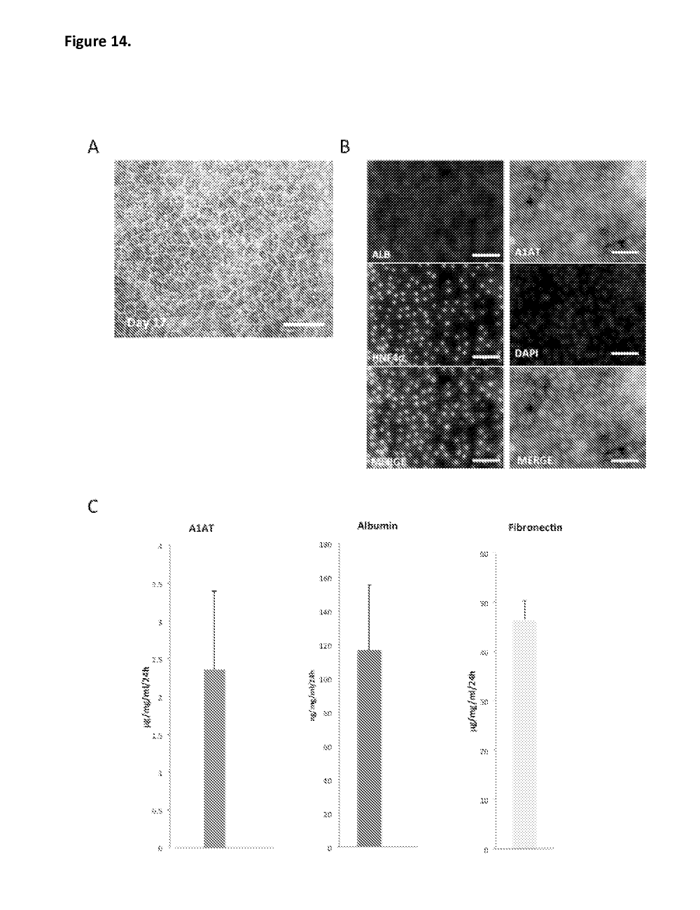

An important attribute of any differentiation methodology is the ability to translate it to other lines. This is especially important in the case of hiPSCs, as these provide the basis to model hepatic disease and potentially provide personalised medicine. In addition, the ability to derive hepatocytes of a defined genotype is of utility in the areas of toxicology and drug development. Two other hESC lines, 207 and 360, were tested using the conditions applied to the hESC line H1. It was observed that Phase I of the procedure was very inefficient and did not produce the predicted morphology as observed in FIG. 2B. A titration of the small molecule CHIR99021 (1-10 μM) in RPMI-B27±insulin was performed. The optimal concentration of CHIR99021 was 4 μM in RPMI-B27 minus insulin in the case of both 207 and 360. After performing the differentiation both lines efficiently generated smHLCs as assessed by morphology (FIGS. 10C and 14A). The 207 smHLCs were also shown to store glycogen by PAS staining (FIG. 10C), and express a panel of hepatic markers (HNF4A, AFP, ALB, A1AT, APOA2 and FIBRIN) by RT-PCR. The expression levels of the hepatocyte markers ALB, HNF4A and alpha-1-antitrypsin (SERPINA1) were assessed by TaqMan RT-qPCR (FIG. 10D), and found these to be very similar to those observed for H1 derived HLCs (FIG. 5A). Both 207 and 360 lines also demonstrated extensive expression of albumin, AFP, HNF4α and alpha-1-antitrypsin by immunofluorescence (FIGS. 10B and 14B). It was next assessed if 207 and 360 smHLCs secreted albumin, alpha-1-antitrypsin and fibronectin by ELISA. All three proteins were detected in the medium at levels similar to those observed for H1 (FIGS. 10E and 14C). Thus it was demonstrated that the protocol was applicable to several hESC lines.

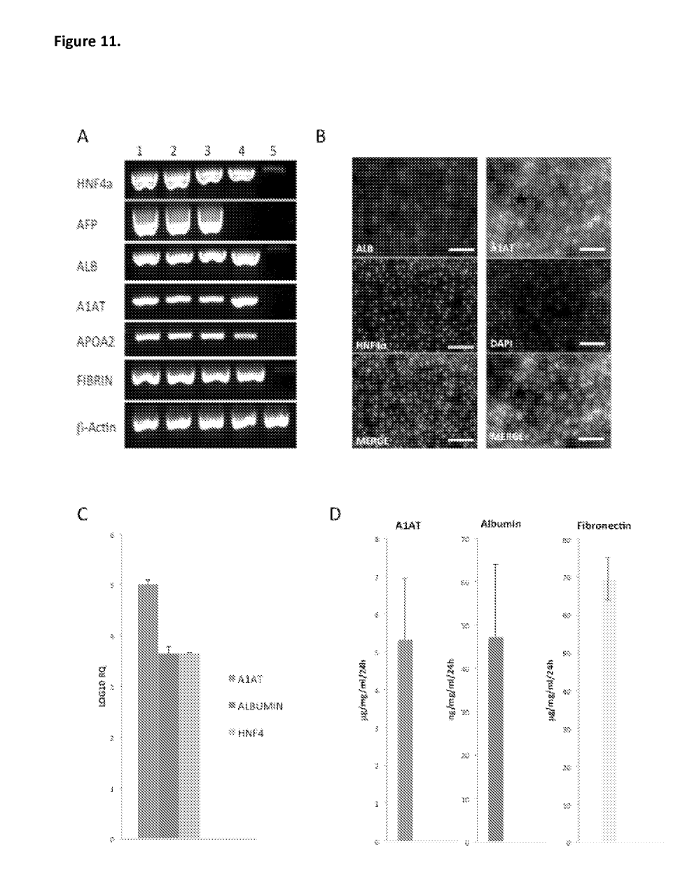

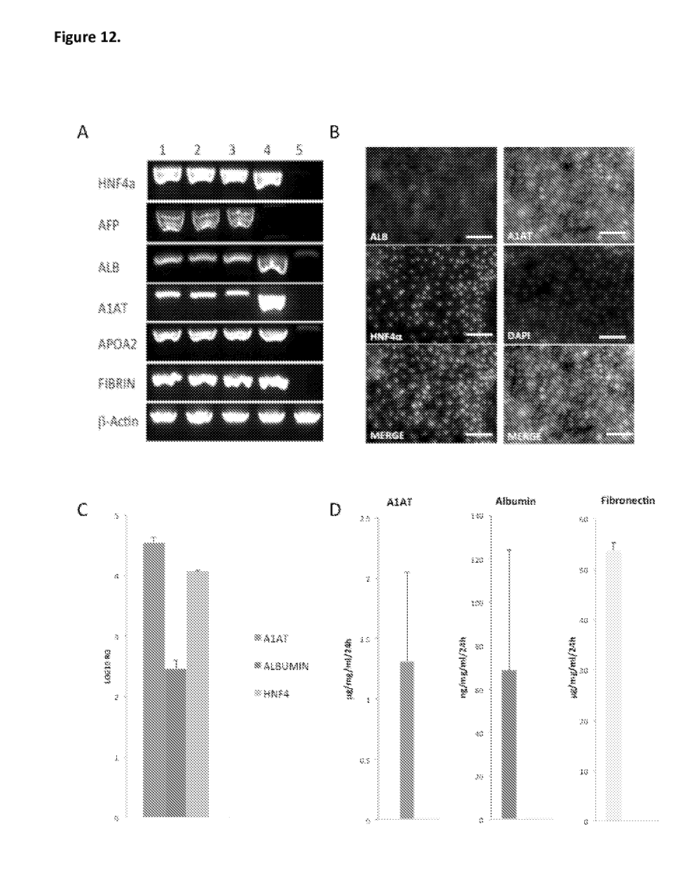

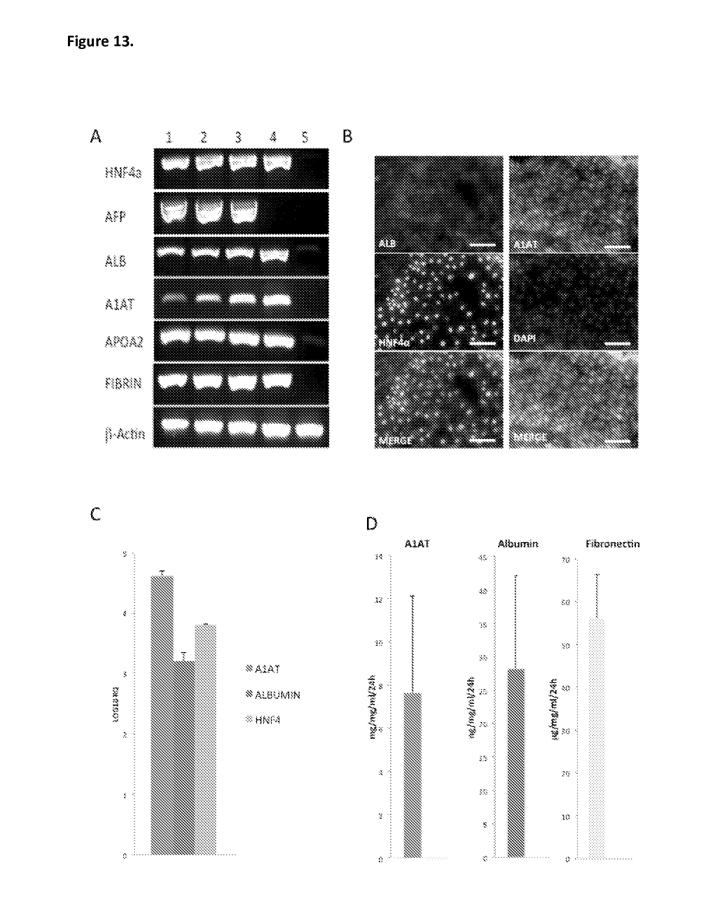

It was next assessed if this was applicable for differentiation of hiPSCs. Three different hiPSC clones derived from the fibroblast line Detroit were tested. A preliminary screen for Phase I of the differentiation (as described above) was performed and it was demonstrated that in all cases 4 μM CHIR99021 and RPMI-B27 minus insulin produced the most satisfactory results. The hiPSCs lines Detroit A, B and C were then subjected to the small molecule driven procedure and were assessed for the ability to form smHLCs. The three hiPSC lines responded to the small molecule procedure, and in all cases the hiPSCs produced cells with typical hepatic morphology. They all expressed the hepatic markers HNF4A, AFP, ALB, A1AT, APOA2 and FIBRIN as assessed by RT-PCR FIGS. 11A, 12A and 13A). The expression levels of the hepatocyte markers ALB, HNF4A and alpha-1-antitrypsin (SERPINA1) were compared by TaqMan RT-qPCR (FIGS. 11C, 12C and 13C), and found similar levels to those observed in the hESC lines H1 and 207 (FIG. 5A and FIG. 10D). It was demonstrated hiPSC derived smHLCs were derived via definitive endoderm by showing CYP7A1 was expressed (40) (FIG. 9B). All lines demonstrated extensive expression at the protein level of albumin, AFP, HNF4α and alpha-1-antitrypsin as assessed by immunofluorescence (FIGS. 11B, 12B and 13B). Finally, it was demonstrated the production of the serum proteins albumin, alpha-1-antitrypsin and fibronectin by ELISA, at levels similar to their hESC counterparts (FIGS. 6D, 7D and 8D).

smHLCs can be Differentiated in a 3D Scaffold

Differentiation of the hESC line H1 and the hiPSC line Detroit was performed in a 3D environment by encapsulating the cells in an alginate scaffold. The cells were cultured using the same conditions as in 2D with the exception that the spheres were cultured for 6 days in human ES media, then transferred to Phase I conditions (RPMI-B27+Chir). All pluripotency marks were maintained as assessed by TaqMan, to the point prior to transition to Phase I. The ability of hPSCs to be directed to definitive endoderm (DE) in 3D was assessed via the DE marks SOX17 and FOXA2. Significant induction of these marks in 3D as compared to 2D differentiation was observed, an order of magnitude induction of both SOX17 and FOXA2 over 2D. Further differentiation through phases II and III is performed as described above.

### Example 2

### Experimental Procedures

Cell Culture.

H1 hESCs (WiCell) and 207 hESCs (Ström et al., 2010) and Detroit 551 (ATCC CCL-110) hiPSCs were maintained at 37° C./5% CO₂ in feeder-free conditions using Growth Factor Reduced Matrigel (Sigma-Aldrich) and E8 Medium (Life Technologies), with routine passaging performed at a 1:3 ratio using 0.5 mM ethylenediaminetetraacetic acid (EDTA) (Life Technologies). All Matrigel plates were coated with a 1:48 dilution in Advanced DMEM-F12 (Life Technologies) and incubated at 37° C./5% CO₂ for 1 hour prior to use.

Hepatocyte Differentiation.

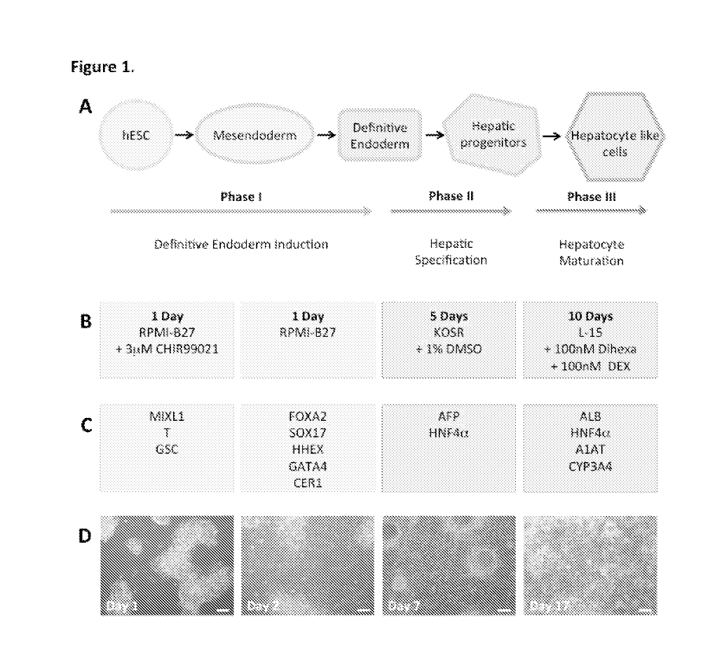

Cells were seeded onto Matrigel coated 12 well plates in E8 medium at a 1:3-1:4 split ratio, and allowed to adhere for 24 hours at 37° C./5% CO₂. It should be noted the optimal cell density for each line needs to be established. The cells were washed with phosphate buffered saline (PBS) before being treated with differentiation media. The differentiation protocol utilises similar base media to a well-established differentiation protocol (Hay et al., Proc Natl Acad Sci USA 105, 12301-12306 2008), with replacement of all growth factors with small molecules, and minor adjustments in timing. The protocol incorporates several stages (FIG. 1) utilising different small molecules.

Phase I of differentiation consists of a 24 hour treatment with RPMI-B27±insulin (RPMI 1640 GlutaMAX+B27 supplement, both from Life Technologies) plus 3-4 μM CHIR99021 (Stemgent), followed by a 24 hour treatment with RPMI-B27 alone. Phase II consists of 5 days of treatment with Knockout DMEM containing 20% Knockout Serum Replacement, 2 mM GlutaMAX, 100 μM 2-mercaptoethanol, 1×MEM non-essential amino acids (Life Technologies) and 1% DMSO (Sigma-Aldrich). Phase III consists of 10 days of treatment with Leibovitz L-15 media containing 8.3% tryptose phosphate broth, 10 μM hydrocortisone 21-hemisuccinate, 50 μg/ml sodium-L-ascorbate, 100 nM dexamethasone (all from Sigma-Aldrich), 0.58% insulin-transferrin-selenium (ITS), 2 mM GlutaMAX (all from Life Technologies), 8.3% foetal bovine serum (Lonza), and 100 nM Dihexa (kind gift from Prof. Joseph Harding, Washington State University). During Phases II and III cells are fed every 48 hours. Cells were photographed during differentiation using a Zeiss phase contrast microscope and ZEN software. The scale bars represent 100 μm.

Control H1 hESCs (Growth Factor) were differentiated using activin A, Wnt3a, DMSO, OSM and HGF (Peprotech) as described previously (Hay et al., 2008, supra: Sullivan et al., Hepatology 51, 329-335 2010). All small molecules were made up in DMSO; vehicle control differentiations were performed using equivalent concentrations.

Small Molecules.

For a complete list of small molecules, sources and purity see Table 4.

Human Induced Pluripotent Stem Cell Derivation and Characterisation.

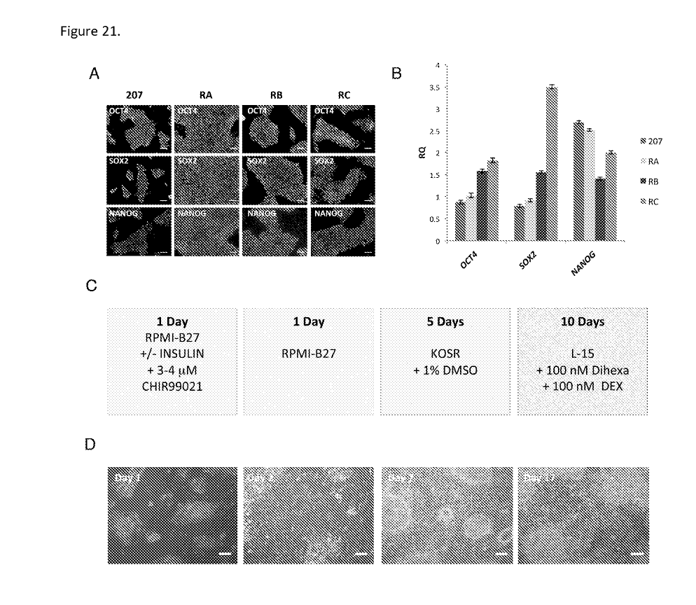

Detroit 551 fibroblasts were obtained from the American Type Culture Collection (ATCC CCL-110), hOCT4, hSOX2, hcMYC, and hKLF4, retrovirus viral particles were generated by Vectalys and transduced at an MOI of 5 as described by Vallier and colleagues (Vallier et al., 2009). On appearance, hiPSCs were picked and expanded feeder free on Matrigel (Sigma-Aldrich) in E8 Medium (Life Technologies). It was verified that the iPSC lines expressed human embryonic stem cell markers by immunocytochemistry for NANOG, SOX2 and OCT4 expression (FIG. 21A). RT-qPCR was used to confirm that iPSCs expressed NANOG, SOX2 and OCT4 (FIG. 211B) and that they had silenced the exogenous genes that were used for reprogramming. The iPSC lines were karyotyped using KaryoLite BoBs (Perkin Elmer) and demonstrated they were normal (performed by Finnish Microarray and Sequencing Centre (FMSC)). Finally, it was demonstrated that the derived hiPSCs were able to generate all three germ layers: ectoderm (neurons), mesoderm (cardiomyocytes) and endoderm (hepatocytes) (data not shown), indicating that the iPSCs generated are pluripotent.

RNA Isolation and RT-qPCR.

RNA was isolated from cells using TRIzol according to manufacturer's instructions and quantified using a spectrophotometer (NanoDrop). cDNA was prepared using the High Capacity Reverse Transcription kit and a thermal cycler (both from Life Technologies). RT-qPCR was performed using a TaqMan ViiA7 Real Time PCR System with TaqMan Gene Expression Master Mix (Life Technologies). TaqMan assays were used to assess markers of interest and ACTB was used as an endogenous control (Life Technologies); see Table 1 for details. Expression levels were quantified relative to ACTB and normalised to undifferentiated pluripotent control samples or definitive endoderm cells as specified. Results are shown as the mean of 3 independent experiments; error bars represent standard deviation.

Immunofluorescence.

Cells were washed with PBS before being fixed with a 10 minute treatment of ice cold methanol. Fixed cells were washed in 0.1% PBS-T: PBS containing 0.1% Tween 20 (Sigma-Aldrich). Cells were blocked for 1 hour in 10% normal goat serum (Life Technologies) made up in 0.1% PBS-T. Cells were then washed twice before being treated with primary antibodies overnight at 4° C.; see Table 2 for antibody details. All primary antibodies were made up in 1% normal goat serum in 0.1% PBS-T. Secondary antibody only controls were also included. Following primary incubations, all cells were washed twice and treated with Alexafluor secondary antibodies (Life Technologies) for 1 hour at room temperature. The secondary antibodies were made up in PBS. Cells were then washed twice in PBS-T and twice in PBS before being mounted using Fluoroshield with DAPI (Sigma-Aldrich) and glass coverslips. Cells were imaged using a Zeiss Observer Fluorescence Microscope and Axiovision imaging software. The scale bars represent 100 μm.

Glycogen Storage, Periodic Acid—Schiff Staining Assay and Uptake of Indocyanine Green.

In order to assess glycogen storage, differentiated cells were fixed and treated with a periodic acid-Schiff staining kit (Sigma-Aldrich) in accordance with manufacturer's instructions and imaged using a Zeiss phase contrast microscope and ZEN software. The scale bars represent 100 μm. The cellular uptake of indocyanine green was assessed. Briefly, ICG (Sigma-Aldrich) was reconstituted in water and used at a final concentration of 1 mg/ml. Cells were incubated in media supplemented with ICG for 1 hour, the cells were then washed with PBS and imaged using a Zeiss phase contrast microscope and ZEN software.

Cytochrome P450 Induction and Analysis.

Induction of cytochrome P450 activity was assessed in both small molecule and growth factor derived HLCs. CYP1A2 activity was detected using the P450-Glo CYP1A2 Induction/Inhibition Assay kit (Promega, Cat. no. V8422). CYP3A4 activity was detected using the P450-Glo CYP3A4 (Luciferin-PFBE) Cell-Based/Biochemical Assay (Promega V8902). Assays were performed according to the manufacturer's instructions for non-lytic P450-Glo assays using cultured cells in monolayers. Cytochrome P450 inductions were performed using the following inducers: for CYP3A4, Rifampicin (25 μM) and CYP1A2, Omeprazole (100 μM) (both purchased from Sigma). Briefly, cells were cultured to day 20 of the differentiation protocol and Rifampicin or Omeprazole was added to L-15 culture medium, which was formulated as described above, but without dexamethasone or hydrocortisone. Medium was replaced daily for 72 hours. After 72 hours, the cells were washed 4 times with PBS (calcium/magnesium free) and then assayed. Briefly, for CYP1 A2 the substrate Luciferin-1A2 was diluted to 6 μM in PBS (calcium/magnesium free) containing 3 mM freshly prepared salicylamide (Sigma). The Luciferin substrate was added to each well (1 ml per well of a 6 well plate), and incubated for 60 minutes followed by detection. For CYP3A4, the substrate Luciferin-PFBE was diluted in L-15 culture medium (formulated as described above), to a final concentration of 50 μM. After washing the cells, 1 mL of Luciferin-PFBE containing media was added to each well and incubated for 4 hours followed by detection. Basal activity was assessed as above with the omission of inducers. In addition, no cell media controls were included. Finally as a negative control, the pluripotent hESC line H1 was assayed for CYP activity as described above. All data was normalised to total protein content in each well. Data is presented as the mean values of 6 independent experiments; error bars represent standard deviation.

Serum Protein Production.

Cells were incubated for 24 hours in 1 ml of media. ELISA kits were then used to detect human albumin (Alpha Diagnostics), fibronectin (AbCam) and alpha-1-antitrypsin (AbCam) in the supernatants according to manufacturer's instructions. Negative control incubations without cells were included as blanks. Results are normalised to protein weight, and given as the mean of 3 independent experiments; error bars represent standard deviation.

Protein Extraction and Quantification.

Cells were lysed in 250 μl of SUMO buffer containing 2% sodium dodecyl sulphate (SDS), 50 mM Tris (pH 8), 1 mM EDTA and 10 mM iodoacetamide (Sigma-Aldrich) for 5 minutes at room temperature. Total protein was quantified using a BCA Assay Kit (Pierce) and an absorbance plate reader (Tecan).

PCR and Gel Electrophoresis.

PCR was carried out using AmpliTaq Gold 360 Master Mix (Life Technologies) supplemented with the relevant oligonucleotide pairs. All assays were run against an ACTB control to ensure equivalent amounts of input cDNA; in all cases 5 ng of input cDNA was used. The oligonucleotide sequences are provided in the Table 3. The PCR products were resolved using agarose gel electrophoresis.

Cell Counting.

Immuno-stained cells were quantified for expression of stage specific markers by manual counting. For Phase I, FOXA2 and SOX17 were counted separately. For Phase II, cells were scored positively if the nucleus was stained for HNF4A and the cytoplasm was stained for AFP. For Phase III, cells were scored positively if the nucleus was HNF4A positive and the cytoplasm was ALB positive. Phase III cells were also quantified for AFP and A1AT staining and were counted as positive if the cytoplasm was stained. In all cases, 3D areas in the image were excluded due to difficulties in counting the nuclei. A minimum of 10 fields of view were quantified, with a minimum of 250 cells counted per field of view. Percentages are presented as the average of all field quantifications, plus or minus the standard deviation across all fields.

Statistical Analysis.

Results were evaluated by performing t tests. p<0.09 was determined significant.

### Results

Production of Definitive Endoderm (DE) Using GSK-3 Inhibition (Phase I).

The ability to produce hepatocytes from hPSCs that have utility in both clinical and research arenas utilizes methodologies that are robust in terms of efficiency and reproducibility. The majority of methodologies to date are far from this and also reliant on recombinant growth factors to direct cellular fate. This will prove a major hurdle if these cells are to be utilised in a therapeutic environment. A differentiation procedure that is devoid of growth factors and driven by small molecules is described herein. The procedure is notionally trisected into 3 phases inducing DE differentiation (Phase I), hepatic specification (Phase II) and hepatocyte maturation (Phase III).

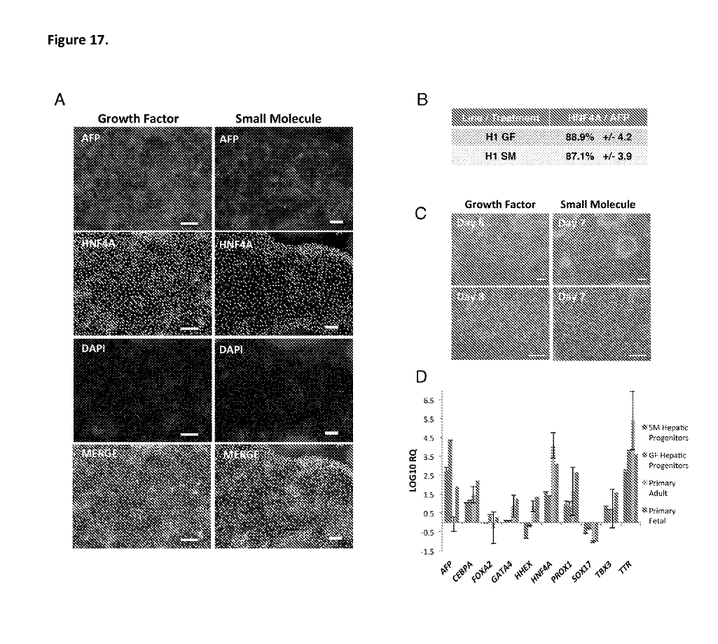

Following studies of the utility of GSK-3 inhibition in priming pluripotent cells for endodermal differentiation (Tahamtani et al., Stem Cells Dev 22, 1419-1432 2013), and reports that Wnt/β-Catenin signalling regulates SOX17 expression and is essential for endoderm formation (Engert et al., Development 140, 3128-3138 2013), experiment were performed to establish whether this approach was suitable for the generation of functional hepatocytes. The conditions for DE differentiation were established in the hESC line H1. Through a 24 hour treatment with 3 μM CHIR99021, followed by 24 hours of non-directed differentiation in RPMI-B27, it was possible to guide hPSCs through developmentally relevant stages to produce a population of DE (FIG. 16A). Over a 48 hour period dynamic changes in the gene expression pattern were observed (FIG. 7). By 48 hours elevated expression of DE markers such as FOXA2, GSC, SOX17, HHEX and CER1 (Ang et al., Development 119, 1301-1315 1993; Blum et al., Cell 69, 1097-1106 1992; Kanai-Azuma et al., Development 129, 2367-2379 2002; Monaghan et al., Development 119, 567-578 1993; Sasaki and Hogan, Development 118, 47-59 1993) were present. In addition, early events of the differentiation indicated transition through a primitive streak (PS) intermediate. A rapid upregulation of NODAL was observed within 4 hours of exposure to CHIR99021, which is indicative of a transition towards a PS population (FIG. 7) (Lu et al., Curr Opin Genet Dev 11, 384-392 2001). This was followed by induction of the PS markers T and GSC (FIG. 7). The markers SOX17, GSC, FOXA2 and MIXL1 are expressed in extra-embryonic endodermal lineages as well as DE. In order to demonstrate that the differentiation procedure was not producing primitive endoderm, the levels of SOX7 were assayed. No upregulation was observed during the procedure (FIG. 17A). The observed patterns of expression are similar to those seen with a 3 day treatment of activin A and Wnt3a or a 5 day treatment of activin A (D'Amour et al., Nat Biotechnol 23, 1534-1541 2005; Hay et al., 2008, supra). These changes in gene expression were accompanied by morphological changes; the cells shifted from a pluripotent morphology to dense, bright clusters at 24 hours, followed by a petal like morphology at 48 hours (FIG. 16B). At the 48 hour time-point (Phase I endpoint), co-expression of FOXA2 and SOX17 were observed at the protein level using immunofluorescence (FIG. 16C). Treatments with activin A/Wnt3a, CHIR99021 and vehicle control were compared and equivalent co-expression of the DE proteins FOXA2 and SOX17 was observed by immunofluorescence in the growth factor and small molecule treated cells (FIG. 16C) and no co-expression in the control. Next it was assessed if GSK-3 inhibition was a generic mechanism to drive hPSCs to DE. FIG. 16C demonstrates the utility of an alternative GSK-3 inhibitor (BIO—1 μM) to produce FOXA2/SOX17 positive cells under the same conditions, indicating that GSK-3 inhibition followed by its removal is responsible for commitment to DE. BIO and CHIR99021 are potent pharmacological GSK-3 specific inhibitors that result in activation of the Wnt signalling pathway (Sato et al., Nat Med 10, 55-63 2004; Sineva and Pospelov, Biol. Cell 102, 549-560 2010), so the ability of the protein Wnt3a alone to drive differentiation towards DE was assessed. FIG. 16C demonstrates that treatment with Wnt3a was sufficient to produce populations of cells that expressed the DE markers FOXA2 and SOX17. This observation indicates that Wnt3a treatment alone can facilitate the production of DE, and that the inclusion of activin A is not necessary for DE production in vitro. All treatments described gave similar efficiencies with respect to FOXA2 and SOX17 positive cells, 78%-85% for FOXA2 and 79%-87% for SOX17 (FIG. 16D).

Hepatic Specification Through DMSO Treatment of Definitive Endoderm (Phase II).

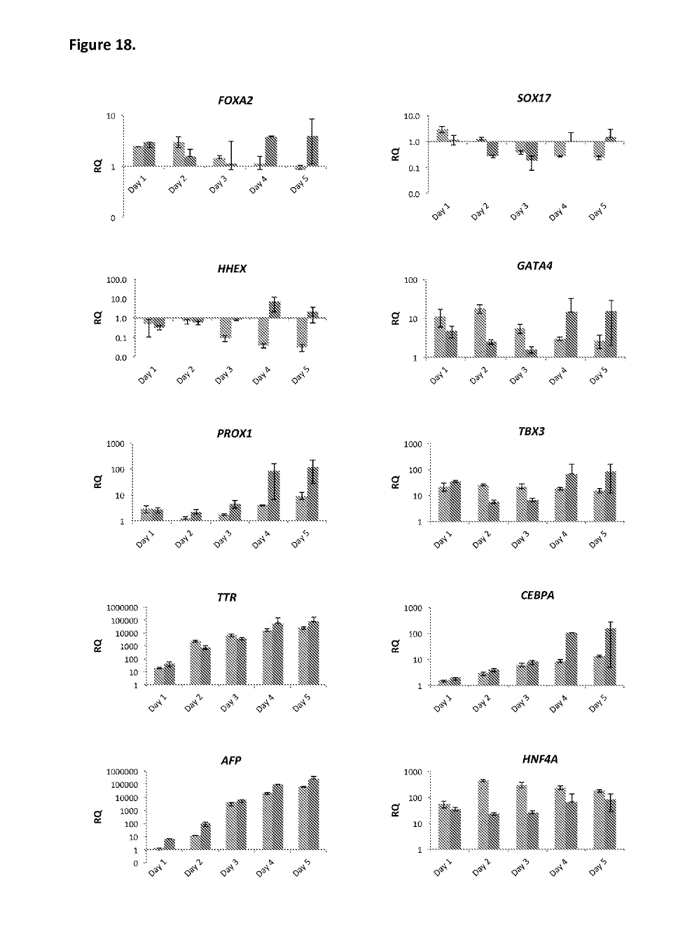

Following the production of DE through small molecule stimulation, the next step was to specify a hepatic fate. Routes to efficiently produce an AFP/HNF4A positive hepatic progenitor population were tested. There are reports of the utility of the small molecule DMSO in stem cell differentiation and specifically in the generation of hepatic progenitors (Hay et al., 2008, supra; Rambhatla et al., Cell Transplant. 12, 1-11 2003; Soto-Gutierrez et al., Cell Transplant. 15, 335-341 2006; Sullivan et al., 2010, supra). Therefore, a 5 day treatment with 1% DMSO was assayed. On subjecting DE to Phase II conditions (DMSO) a rapid change in morphology and a spurt of proliferation was observed. After the 5 days of treatment 87% co-expression of AFP and HNF4A, as assessed by immunofluorescence, was observed (FIG. 17A-B). This was comparable to activin A/Wnt3a followed by DMSO treatment, in line with previous reports (FIG. 17A-B). In addition the cells exhibited typical hepatocyte progenitor morphology as assessed by phase contrast microscopy (FIG. 17C). The levels of AFP/HNF4A co-expression observed are indicative that this phase of the differentiation is extremely efficient (FIG. 17B). Gene expression was analysed after the end of hepatic specification by RT-qPCR (FIG. 17D), the panel shows a repertoire of hepatic progenitor markers that were expressed including AFP, CEBPA, FOXA2, GATA4, HNF4A, PROX1, TBX3 and TTR. To assess the differentiation of DE to hepatic progenitors (hepatoblasts), the gene expression levels of several key developmental markers, which are known to regulate hepatoblast formation in vivo, during the 5 day Phase II protocol, were monitored (Si-Tayeb et al., Hepatology 51, 297-305 2010a). It was observed that both small molecule and growth factor derived DE (hESC line H1) followed a similar trajectory towards hepatic progenitors (FIG. 18). During differentiation to hepatic progenitors maintenance of FOXA2 levels and an induction of GATA4, both of which are known to be pioneer factors critical to promote the hepatic gene expression program, were observed (Kaestner, Cell Cycle 4, 1146-1148 2014). Over the 5 days there was an increase in expression of PROX1 and TBX3, which are believed to interact in vivo to promote the migration and proliferation of hepatoblasts from the primary liver bud (Sosa-pineda and Wigle, Prox1 25, 254-255 2000; Lüdtke et al., Hepatology 49, 969-78 2009). Furthermore, several key regulators and markers of hepatocyte differentiation were assayed and strong induction of CEBPA, HNF4A, TTR, and AFP was observed.

Production of HLCs Via Dexamethasone and the HGF Receptor Agonist Dihexa (Phase III).

The final stage of HLC differentiation (hepatic maturation) has been performed using a wide range of growth factors such as HGF, OSM, FGF4, VEGF and EGF (Songyan Han and Valerie, J. Stem Cell Res. Ther. 2012). N-hexanoic-Tyr, Ile-(6) aminohexanoic amide (Dihexa) and the small molecule glucocorticoid mimetic dexamethasone (DEX were assayed in the maturation step.

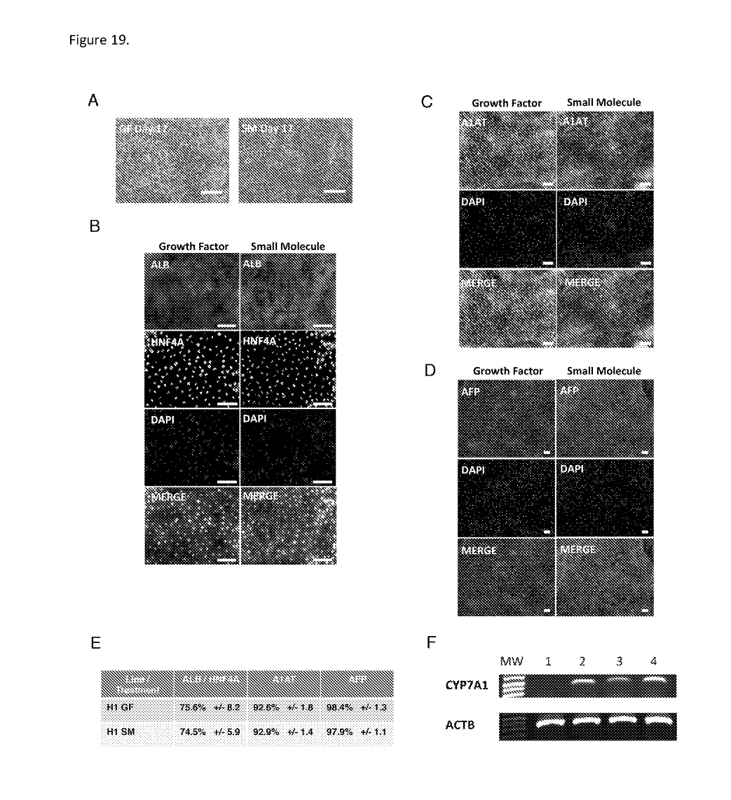

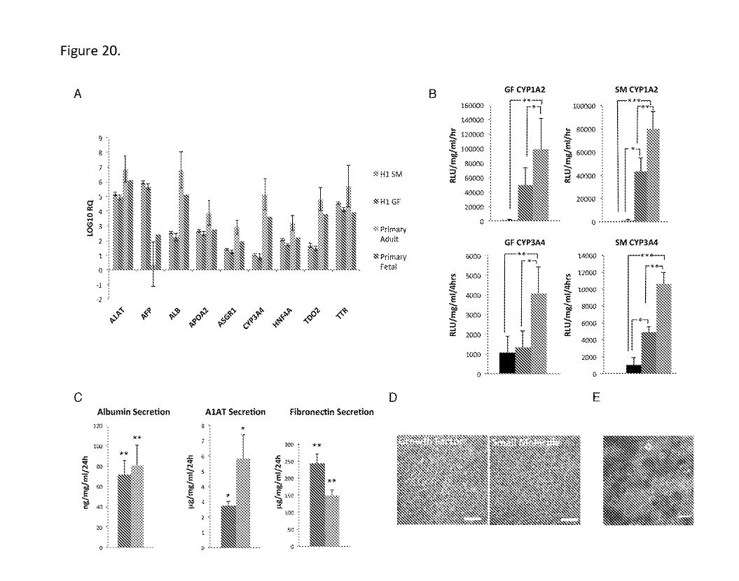

A number of base media were tested to establish the optimal concentrations of DEX and Dihexa. The media HepatoZYME (Life Technologies) was used to establish the optimal concentrations of DEX and Dihexa as being 100 nM for each. William's base medium was next assessed. Both DEX and Dihexa were required and the above concentrations gave the best results in terms of morphology and function. A modified formulation of Leibovitz L-15 medium (L-15), which has been described as a standard method to generate mature hepatocytes (Hay et al., 2008, supra; Sullivan et al., 2010, supra) was utilized for further experiments. L-15 medium was supplemented with DEX and Dihexa (both at 100 nM), which led to the production of cells displaying typical hepatocyte morphology at the endpoint of the small molecule driven differentiation protocol (FIG. 19A). The cells were large and angular with bright junctions and in some instances contained multiple nuclei. The resulting HLCs demonstrated expression of the hepatocyte markers albumin (ALB), HNF4A, alpha-1-antitrypsin (A1AT) and AFP by immunofluorescence (FIG. 19B-D). Comparable data can be seen for growth factor based differentiation in FIG. 19B-D. Comparable efficiencies of differentiation were observed between the growth factor and small approaches by assessing ALB/HNF4A, A1AT and AFP (FIG. 19E). To corroborate that the source of the HLCs is from DE rather than yolk sac, it was demonstrated that the DE origin marker CYP7A1 was expressed (Asahina et al., Genes Cells 9, 1297-1308 2004) (FIG. 19F). To ensure that the protocol was equivalent to previously described growth factor based methods, functional hepatocytes were produced as described by Hay and colleagues (Hay et al., 2008, supra) (see FIG. 19B-D) for validation. Gene expression was analysed by RT-qPCR (FIG. 20A), the panel shows a repertoire of hepatic markers that were expressed: A1AT (SERPINA1), AFP, ALB, APOA2, ASGR1, CYP3A4, HNF4A, TDO2 and TTR. The observed relative levels of expression were very similar irrespective of being derived via the growth factor or small molecule based protocol (FIG. 20A). Similar levels of expression were observed with respect to fetal hepatocytes for A1AT (SERPINA1), APOA2, ASGR1, HNF4A and TTR. However, higher levels of expression of AFP were observed for both small molecule and growth factor derived HLCs compared to adult and fetal hepatocytes. In all cases, except for AFP, higher levels of expression of all hepatic markers were observed in primary adult hepatocytes.

Small Molecule Derived HLCs Demonstrate Hepatic Function.

It was next assessed if smHLCs displayed functional hepatic characteristics. An important function of hepatocytes is the ability to clear xenobiotics via metabolism through the cytochrome P450 iso-enzymes. smHLCs were assessed for their metabolic potential, as compared to growth factor derived HLCs. The cytochrome P450 enzymes (CYP) are critical in drug metabolism, in particular CYP1A2 and CYP3A4. The function of these CYPs was assessed in terms of their basal activity and their ability to be induced by Rifampicin (CYP3A4) and Omeprazole (CYP1A2). Higher basal CYP activity was observed in the smHLCs as compared to hESC H1 controls for both CYP1A2 and 3A4 (FIG. 20B). On challenge with known inducers of these enzymes significant induction of both CYPs, to a similar degree as in growth factor derived HLCs, was observed (FIG. 20B). Another key function of hepatocytes is the production of serum proteins, so the ability of the smHLCs to secrete albumin, alpha-1-antitrypsin and fibronectin was assayed by ELISA. All three proteins were detected in the medium at levels similar to those seen from growth factor derived cells (FIG. 720). Another function tested was the ability to store glycogen. smHLCs were stained with periodic acid—Schiff (PAS), and counterstained with hematoxylin and eosin. Extensive cytoplasmic staining (pink to purple), indicative of glycogen storage, was observed at levels similar to those observed in growth factor derived hepatocytes (FIG. 20D). The uptake of indocyanine green (ICG) in smHLCs was observed; after a brief treatment, ICG positive cells were clearly visible (FIG. 20E).

smHLCs can be Derived from Multiple Human Pluripotent Stem Cell Lines.

An important attribute of any differentiation methodology is the ability to translate it to other cell lines. This is especially important in the case of hiPSCs, as these will provide the basis to model hepatic disease and potentially lead to the development of personalised medicine. In addition, the ability to derive hepatocytes of a defined genotype is of utility in the areas of toxicology and drug development. To test this the potential of a number of human pluripotent stem cell lines was assessed. One additional hESC line (207) and 3 different hiPSC clones derived from the fibroblast line Detroit 551 (RA, RB, RC) were assayed using the conditions applied to the hESC line H1.

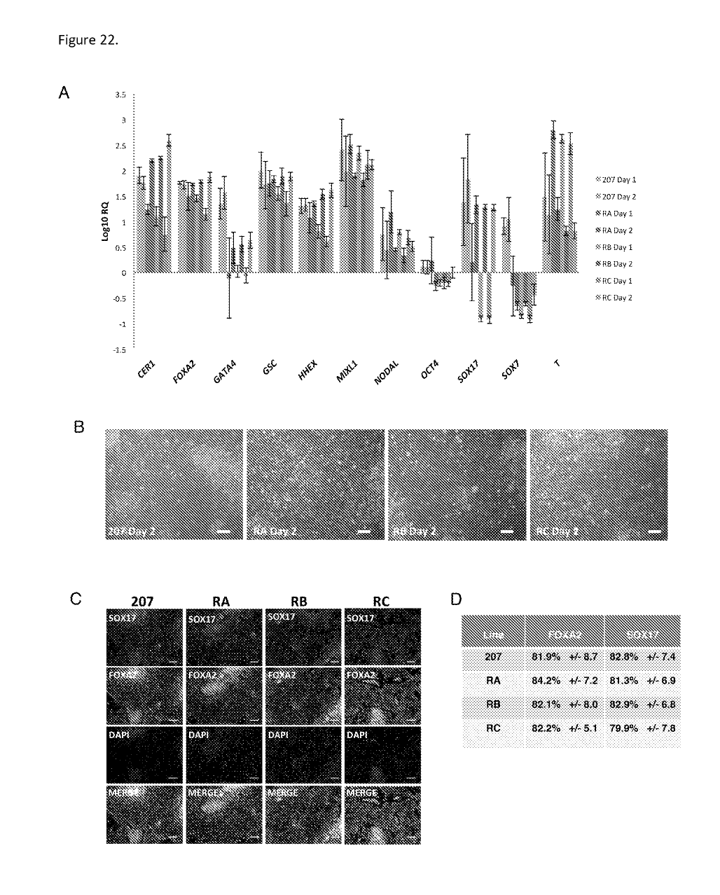

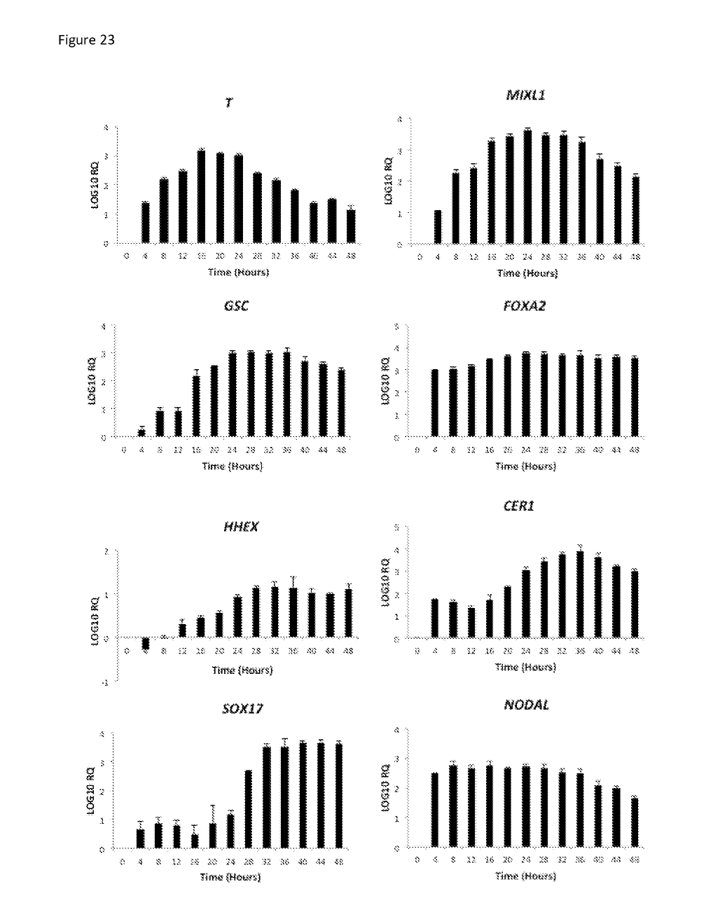

Through an initial 24 hour treatment with 3 μM CHIR99021, followed by 24 hours of non-directed differentiation in RPMI-B27, differentiation was very inefficient and did not produce the predicted morphology as previously observed for the hESC line H1 (FIG. 16B). A titration of the small molecule CHIR99021 (1-10 μM) in RPMI-B27±insulin was performed (FIG. 21C). The optimal concentration for CHIR99021 was 4 μM in RPMI-B27 minus insulin in all cases. Over a 48 hour period, dynamic changes in the gene expression pattern were observed (FIG. 22A) similar to those previously observed for the hESC line H1. On completion of Phase I (48 hours) elevated expression of DE markers such as FOXA2, GSC, SOX17, HHEX and CER1 was observed. These changes in gene expression were accompanied by morphological changes consistent with the hESC line H1 differentiation above (FIG. 22B). At the 48 hour time-point (Phase I endpoint), co-expression of FOXA2 and SOX17 was observed at the protein level using immunofluorescence (FIG. 22C). All lines gave similar efficiencies with respect to FOXA2 and SOX17 positive cells, 81%-84% for FOXA2 and 79%-82% for SOX17 (FIG. 22D). To ensure that hiPSCs were following a developmentally relevant route the timing of key events in DE specification was assayed. FIG. 23 shows induction of the PS markers T, GSC and NODAL in the first 24 hrs. This is followed by specification to definitive endoderm indicated by robust induction of SOX17, GSC, FOXA2 and MIXL1 (FIG. 23) and no significant upregulation of SOX7 (FIG. 22A).

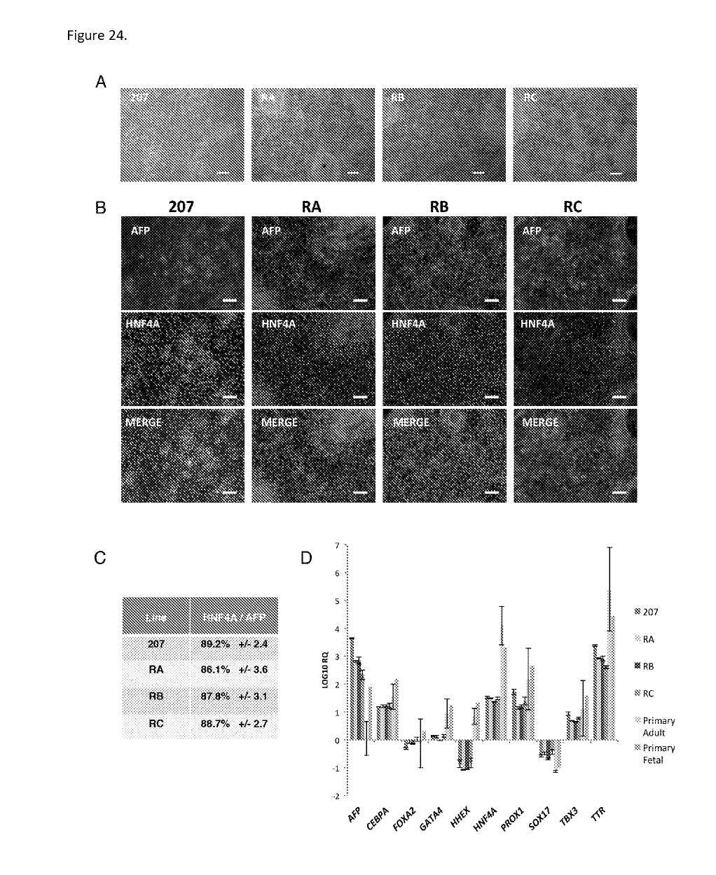

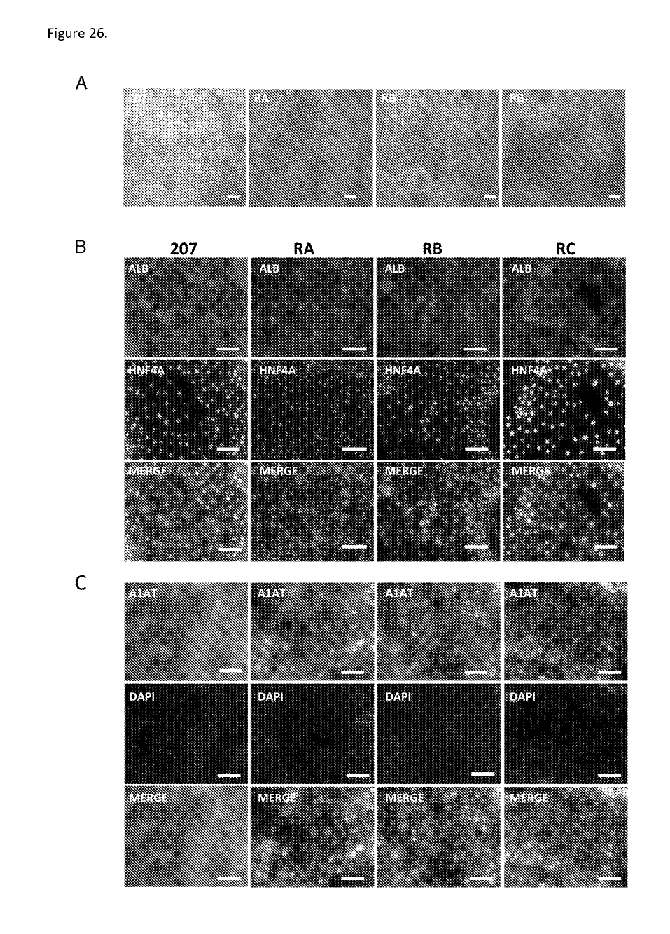

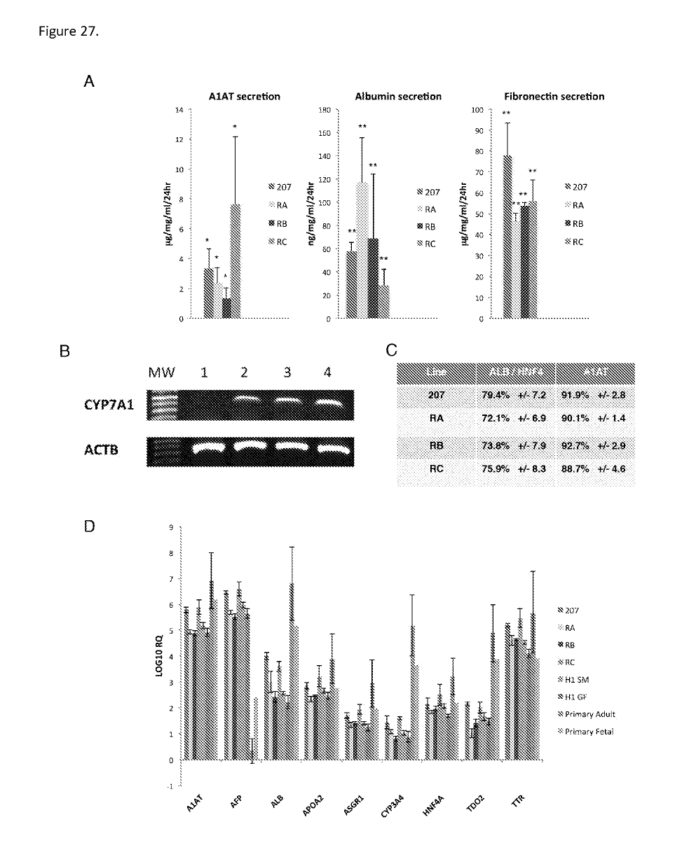

On confirmation of DE specification a hepatic fate was specified using the same Phase II conditions (DMSO) as described above. After the 5 days of treatment a rapid change in morphology, accompanied with proliferation was observed. For all lines tested typical hepatic progenitor morphology as assessed by phase contrast microscopy was observed (FIG. 24A). Efficiencies of between 86%-89% based on AFP/HNF4A co-expression were seen, as assessed by immunofluorescence (FIG. 24B-C). Gene expression was analyzed after the end of hepatic specification by RT-qPCR, at which point equivalent expression profiles as hESC H1 were seen (FIG. 24D). In addition, as with the hESC line H1, a time course over 5 days was performed and a very similar developmental marker profile was observed. The validated hepatic progenitor cells from the different hPSCs were then subjected to Phase III differentiation (FIGS. 1C and 21C). On completion of the differentiation all lines efficiently generated smHLCs as assessed by morphology (FIG. 26A). All lines demonstrated extensive expression of ALB/HNF4A and alpha-1-antitrypsin by immunofluorescence (FIGS. 26B and 26C). Both hESC line 207 and hiPSC derived smHLCs secreted albumin, alpha-1-antitrypsin and fibronectin as assessed by ELISA. All three proteins were detected in the medium at levels similar to those observed for H1 (FIG. 27A). It was confirmed that the hiPSC derived smHLCs were from a DE origin via the presence of CYP7A1 (FIG. 27B). Comparable efficiency of smHLC production to hESC H1, between 72%-79% for ALB/HNF4A and 88%-92% for A1AT were observed (FIG. 27C). The expression levels of the following hepatocyte markers were assayed: A1AT (SERPINA1), AFP, ALB, APOA2, ASGR1, CYP3A4, HNF4A, TDO2 and TTR by RT-qPCR and very similar profiles were observed to H1 derived smHLCs (FIG. 27C). Thus, it was demonstrated that the protocol was applicable to other hESC and hiPSC lines.

TABLE 1

| Target | Manufacturer | Reference |
|---|---|---|
| OCT4 | Life Technologies | Hs00999634_gH |
| SOX2 | Life Technologies | Hs01053049_s1 |
| NANOG | Life Technologies | Hs04260366_g1 |
| NODAL | Life Technologies | Hs00415443_m1 |
| MIXL1 | Life Technologies | Hs00430824_g1 |
| T | Life Technologies | Hs00610080_m1 |
| GSC | Life Technologies | Hs00906630_g1 |
| SOX7 | Life Technologies | Hs00846731_s1 |
| GATA4 | Life Technologies | Hs00171403_m1 |
| FOXA2 | Life Technologies | Hs00232764_m1 |
| SOX17 | Life Technologies | Hs00751752_s1 |
| HHEX | Life Technologies | Hs00242160_m1 |
| CER1 | Life Technologies | Hs00193796_m1 |
| AFP | Life Technologies | Hs00173490_m1 |
| HNF4A | Life Technologies | Hs00230853_m1 |
| CEBPA | Life Technologies | Hs 00269972_s1 |
| PROX1 | Life Technologies | Hs00896294_m1 |
| TBX3 | Life Technologies | Hs00195612_m1 |
| ASGR1 | Life Technologies | Hs01005019_m1 |
| TDO2 | Life Technologies | Hs00199611_m1 |
| APOA2 | Life Technologies | Hs00952079_g1 |
| ALB | Life Technologies | Hs00910225_m1 |
| CYP3A4 | Life Technologies | Hs00604506_m1 |
| TTR | Life Technologies | Hs00174914_m1 |
| A1AT (SERPINA1) | Life Technologies | Hs01097800_m1 |
| ACTB | Life Technologies | N/A |

TABLE 2

| | | Catalogue | | |
|---|---|---|---|---|
| Target | Manufacturer | Number | Species | Dilution |
| OCT4 | Stemgent | 09-0023 | Rabbit | 1:100 |
| SOX2 | Stemgent | 09-0024 | Rabbit | 1:100 |
| NANOG | Stemgent | 09-0020 | Rabbit | 1:100 |
| FOXA2 | AbCam | ab40874 | Rabbit | 1:1000 |
| SOX17 | AbCam | ab84990 | Mouse | 1:100 |
| AFP | Sigma-Aldrich | A8452 | Mouse | 1:500 |
| HNF4A | Santa Cruz | sc8987 | Rabbit | 1:100 |
| ALBUMIN | Sigma-Aldrich | A6684 | Mouse | 1:500 |
| Alpha-1-antitrypsin | Santa Cruz | sc30121 | Rabbit | 1:50 |
| Alexaflour 468 | Life | A21206 | Donkey | 1:400 |
| anti rabbit | Technologies | | | |
| Alexafluor 488 | Life | A11059 | Rabbit | 1:400 |
| anti mouse | Technologies | | | |
| Alexafluor 594 | Life | A11005 | Goat | 1:400 |
| anti mouse | Technologies | | | |

TABLE 3

| | Forward sequence | Reverse sequence |
|---|---|---|
| Target | 5′-3′ | 5′-3′ |
| ACTB | TCACCACCACGGCCGAGCG | TCTCCTTCTGCATCCTGTCG |
| | (SEQ ID NO: 1) | (SEQ ID NO: 3) |
| CYP7A1 | CTGCCAATCCTCTTGAGTTCC | ACTCGGTAGCAGAAAGAATA |
| | (SEQ ID NO: 2) | CATC (SEQ ID NO: 4) |

TABLE 4

| | | | | Phase of | Final | |
|---|---|---|---|---|---|---|
| Name | Source | Purity | Solvent | Protocol | concentration | |
| CHIR99021 | STEMGENT | >95% | DMSO | Phase I | 3-4 | μM |
| BIO | Tocris | >98% | DMSO | Alternative | 1 | μM |
| | | | | Phase I | | |
| DMSO | Sigma-Aldrich | ≧99.9% | N/A | Phase II | 1% by volume | |
| Dexamethasone | Sigma-Aldrich | ≧97% | DMSO | Phase III | 100 | nM |
| Dihexa | Kind gift of Prof. Joseph | 93% | DMSO | Phase III | 100 | nM |
| | Harding Washington | | | | | |
| | State University | | | | | |

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

1. A method of differentiating pluripotent stem cells, comprising:
sequentially contacting pluripotent stem cells with CHIR99021; DMSO or a DMSO mimetic; and one or more of a glucocorticoid and an HGF mimetic.

2. (canceled)

3. The method of claim 1, wherein said glucocorticoid is dexamethasone (DEX) or hydrocortisone 21-hemisuccinate.

4. The method of claim 1, wherein said HGF mimetic is selected from the group consisting of Ile-(6) aminohexanoic amide (Dihexa), Nle1-AngIV; N-Acetyl-Nle-Tyr-Ile-His; D-Nle-Tyr-Ile; GABA-Tyr-Ile; Nle-Tyr-Ile-His-NH2; D-Nle-X-Ile-NH—(CH2)5-CONH2, where X is any amino acid; Nle1-Tyr2-Ile3-His4-Pro5, Nle1-Tyr2-Ile3-His4, and Nle1-Tyr2-Ile3.

5. The method of claim 1, wherein said glucocorticoid is provided in combination with said HGF mimetic.

6. The method of claim 3, wherein said DEX and Dihexa are administered at a concentration of 1-1000 nm.

7. The method of claim 6, wherein said DEX and Dihexa are administered at a concentration of 10-100 nm.

8. The method of claim 1, wherein said CHIR99021 is contacted with said pluripotent stem cells for 6-120 hours.

9. The method of claim 1, wherein said DMSO or DMSO mimetic is contacted with said pluripotent stem cells for approximately 2-7 days.

10. The method of claim 1, wherein said pluripotent stem cells are human embryonic stem cells or induced pluripotent stem cells

11. The method of claim 1, wherein said method differentiates said pluripotent stem cells into hepatocytes.

12. A kit, comprising:
a) CHIR99021;
b) DMSO or a DMSO mimetic; and
c) one or more of a glucocorticoid and an HGF mimetic.

13. (canceled)

14. The kit of claim 12, wherein said glucocorticoid is dexamethasone or hydrocortisone 21-hemisuccinate.

15. The kit of claim 12, wherein said HGF mimetic is selected from the group consisting of Ile-(6) aminohexanoic amide (Dihexa), Nle1-AngIV; N-Acetyl-Nle-Tyr-Ile-His; D-Nle-Tyr-Ile; GABA-Tyr-Ile; Nle-Tyr-Ile-His-NH2; D-Nle-X-Ile-NH—(CH2)5-CONH2, where X is any amino acid; Nle1-Tyr2-Ile3-His4-Pro5, Nle1-Tyr2-Ile3-His4, and Nle1-Tyr2-Ile3.

16. The kit of claim 12, wherein said components a); b); and c) are provided in separate containers.

17. A differentiated endodermal lineage cell derived by the method of claim 1.

18. The differentiated endodermal lineage cell of claim 17, wherein said cell is selected from the group consisting of hepatocytes, liver, pancreas, lung, colon, and gut cell types, insulin producing cells, cholangiocytes and intestinal cells.

19-22. (canceled)


SEQUENCE LISTING (4 sequences)

SEQ ID NO: 1 - DNA, 19 bp, Artificial
  Synthetic
tcaccaccacggccgagcg

SEQ ID NO: 2 - DNA, 21 bp, Artificial
  Synthetic
ctgccaatcctcttgagttcc

SEQ ID NO: 3 - DNA, 20 bp, Artificial
  Synthetic
tctccttctgcatcctgtgg

SEQ ID NO: 4 - DNA, 24 bp, Artificial
  Synthetic
actcggtagcagaaagaatacatc